(19) United States
(12) Patent Application Publication
(10) Pub. No.: US 2022/0054485 A1
(43) Pub. Date: Feb. 24, 2022

(54) METHODS OF TREATING RIGHT VENTRICULAR FUNCTION IN PULMONARY HYPERTENSION

(71) Applicant: REGENTS OF THE UNIVERSITY OF MINNESOTA, Minneapolis, MN (US)

(72) Inventors: Kurt Prins, Minneapolis, MN (US); Sasha Prisco, Minneapolis, MN (US)

(73) Assignee: REGENTS OF THE UNIVERSITY OF MINNESOTA, Minneapolis, MN (US)

(21) Appl. No.: 17/406,836

(22) Filed: Aug. 19, 2021

Related U.S. Application Data
(60) Provisional application No. 63/068,240, filed on Aug. 20, 2020.

(51) Int. Cl. A61K 31/4985; A61K 9/00; A61K 45/06; A61P 9/12

(52) U.S. Cl. CPC A61K 31/4985; A61K 9/0019; A61P 9/12; A61K 45/06; A61K 9/0056

(57) ABSTRACT

The present invention provides in certain embodiments methods of treating right ventricular dysfunction in pulmonary hypertension or an elevated blood level of IL-6 by administering a composition comprising a GP130 antagonist in a patient in need thereof. The present invention also provides in certain embodiments a method of treating an elevated blood level of IL-6 by administering a first composition comprising a GP130 antagonist in a patient in need thereof.

### RELATED APPLICATION

This application claims priority to U.S. Provisional Application No. 63/068,240 that was filed on Aug. 20, 2020. The content of the application referenced above is herein incorporated by reference in its entirety.

### BACKGROUND

Pulmonary hypertension (PH) is defined as a resting mean pulmonary artery pressure greater that 20 mmHg. PH is classified by the World Health Organization (WHO) into five categories: (1) pulmonary arterial hypertension (PAH), (2) pulmonary hypertension owing to left heart disease, (3) due to chronic lung disease, (4) chronic thromboembolic pulmonary hypertension (CTEPH), and (5) pulmonary hypertension with unclear multifactorial mechanisms. Ryan et al., “The WHO classification of pulmonary hypertension: A case-based imaging compendium,” Pulmonary Circulation, 2012, vol. 2, no. 1, 107-121. Category 1 PAH is due to pulmonary vascular disease. Thenappan et al., “Pulmonary arterial hypertension: pathogenesis and clinical management,” BMJ, 2018:360:j5492. Category 2 PH results from left ventricular (LV) or left-sided valvular disease. Category 3 PH is due to chronic lung disease, hypoxia or both. Category 4 PH (CTEPH) is due to chronic thromboembolic disease. Category 5 PH includes a collection of PH syndromes caused by a variety of disorders, such as hematological disorders and extrinsic compression of the pulmonary artery.

Pulmonary arterial hypertension (PAH) results from obstructive remodeling of the pulmonary vasculature which ultimately causes right ventricular (RV) failure and death. Median survival in PAH is only 5-7 years, and the strongest predictor of mortality is RV dysfunction (RVD). However, the molecular mechanisms underlying RVD in PAH are incompletely understood. This is highlighted by the fact that all PAH therapies only target the pulmonary vasculature, and not the RV directly. Thus, there is a critical and unmet need for therapeutic targets for RVD to compliment current treatments and to improve survival in PAH.

### SUMMARY

The present invention provides in certain embodiments a method of treating right ventricular dysfunction in pulmonary hypertension by administering a first composition comprising (or consisting essentially of) an GP130 antagonist in a patient in need thereof.

The present invention provides in certain embodiments a method of treating an elevated blood level of IL-6 by administering a first composition comprising (or consisting essentially of) an GP130 antagonist in a patient in need thereof.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A and 1B. MCT rats have a severe RVD phenotype while PAB rats have mild RV D. (1A) Quantification of right ventricular hypertrophy (RVH). MCT rats have more RVH than PAB rats. (1B) Quantification of RV function revealed MCT rats have worse RV function than PAB rats. (*) indicates significantly different than PAB as determined by One-way ANOVA and Tukey post hoc analysis.

FIGS. 2A-2D. Activation of the IL6 Pathway, enhanced microtubule stability, and a reduction in JPH2 in the RV but not LV of MCT rats. (2A) Representative Western blots and quantification (2B) of the IL6 pathway, microtubules, and JPH2 in the RV of MCT rats. (2C) Representative Western blots and quantification (2D) of the IL6 pathway, microtubules, and JPH2 in the LV of MCT rats.

FIGS. 3A-3D. Minor activation of the IL6 pathway and slightly enhanced microtubule stability and reduction in JPH2 in the RV but not LV of PAB rats. (3A) Representative Western blots and quantification (3B) of the IL6 pathway, microtubules, and JPH2 in the RV of PAB rats. (3C) Representative Western blots and quantification (3D) of the IL6 pathway, microtubules, and JPH2 in the LV of PAB rats.

FIGS. 4A-4F. IL6 causes microtubule stabilization which promotes t-tubule remodeling due to junctophilin-2 miss-trafficking in isolated cardiomyocytes. IL6 induces microtubule remodeling (4A and 4B) which disrupts JPH2 t-tubule enrichment (4C and 4D) which eventually causes pathological t-tubule remodeling (4E and 4F). (*) indicates significant difference as determined by t-test.

FIGS. 5A-5K: GP130 antagonism blunts RV STAT3 activation, normalizes expression of the MT-JPH2 pathway, restores t-tubule architecture, augments RV function, and improves survival. (5A) Representative Western blots and (5B) quantification of protein abundance in RV extracts from control, MCT-V, and GP130 antagonist rats. Data shown as expression relative to control levels (dashed line)±SEM. N=4 animals per group. Representative images of RV free wall sections stained with Alexa Fluor-488 conjugated wheat germ agglutinin to evaluate the t-tubule architecture in control (5C), MCT-V (5D), and GP130 antagonist (5E) rats. Scale bar 20 μm. (5F) Quantification of t-tubule architecture and organization by TTorg (arbitrary units are TTpower). N=3 animals per group, 7-14 cardiomyocytes analyzed per animal. GP130 antagonism improved RV function as assessed by TAPSE (5G), percent RV free wall thickness change (5H), cardiac output (5I), and cardiac output normalized to body weight (5J). N=9-14 animals per group. GP130 antagonism improved survival compared to the MCT-V group (5K). N=10 control, 20 MCT-V, and 20 GP130 antagonist animals. (*) indicates statistically different from control and (#) indicates statistically different from GP130 antagonist as determined by one-way ANOVA with Tukey post-hoc analysis or Brown-Forsythe and Welch ANOVA with Dunnett post-hoc analysis. For the survival curves, (*) indicates statistically different from control and GP130 antagonist groups as determined by the log-rank (Mantel-Cox) test. AU: arbitrary units; CBB: Coomassie brilliant blue; Con: control; detyr-tub: detyrosinated α-tubulin; GP130 antag: glycoprotein 130 antagonist; JPH2: junctophilin-2; MCT-V: monocrotaline-vehicle; RV: right ventricle; TAPSE: tricuspid annular plane systolic excursion, STAT3: signal transducer and activator of transcription 3; pSTAT3: phosphorylated STAT3; tub: tubulin.

FIGS. 6A-6C: Proteomic analysis of MAPs in RV extracts from control, MCT-V, and GP130 antagonist rats. (6A) PCA plot showing that GP130 antagonism restored the global expression signature of MAPs to a signature similar to control RV. (6B) Heat map of the proteomic analysis of MAPs demonstrating that the global expression pattern of MAPs in the RV of GP130 antagonist rats more closely resembled control than MCT-V. (6C) Pathway analysis of dysregulated MAPs showed an enrichment of proteins involved in mitochondrial dysfunction and oxidative phosphorylation. GP130: glycoprotein 130; MAPs: microtubule-associated proteins; MCT-V: monocrotaline-vehicle; PCA: principal component analysis; RV: right ventricle.

FIGS. 7A-7F: GP130 antagonism reduces RV hypertrophy. Representative images of cardiomyocytes from H&E stained RV free wall sections from control (7A), MCT-V (7B), and GP130 antagonist (7C) rats. Scale bar 10 μm. Cardiomyocyte area quantified in (7D). N=3 control, 2 MCT-V, and 3 GP130 antagonist RV, 21-29 cardiomyocytes measured per animal. GP130 antagonism reduced RV hypertrophy as assessed by (7E) the Fulton index (RV/LV+S) and (7F) RV weight normalized to body weight. N=10 control, 24 MCT-V, and 19 GP130 antagonist rats. (*) indicates statistically different from control and (#) indicates statistically different from GP130 antagonists as determined by one-way ANOVA with Tukey post-hoc analysis or Brown-Forsythe and Welch ANOVA with Dunnett post-hoc analysis. BW: body weight; GP130 antag: glycoprotein 130 antagonist; MCT-V: monocrotaline-vehicle; LV+S: left ventricle plus septum; RV: right ventricle.

FIGS. 8A-8F: GP130 antagonism decreases collagen expression and RV fibrosis. (8A) Representative Western blots and (8B) quantification of collagen I and III protein abundance in RV extracts from control, MCT-V, and GP130 antagonist rats. Data shown as expression relative to control levels (dashed line)±SEM. N=4 animals per group. Representative RV free wall sections stained with trichrome from control (8C), MCT-V (8D), and GP130 antagonist (8E) rats. Fibrosis indicated by areas in blue. Scale bar 20 μm. Percent RV fibrosis quantified in (8F). N=3 control, 2 MCT-V, and 3 GP130 antagonist RV, 3-4 areas of the RV sampled per animal. (*) indicates statistically different from control and (#) indicates statistically different from GP130 antagonist as determined by one-way ANOVA with Tukey post-hoc analysis. CBB: Coomassie brilliant blue; GP130 antag: glycoprotein 130 antagonist; MCT-V: monocrotaline-vehicle; RV: right ventricle.

FIGS. 9A-9F: GP130 antagonism does not alter the pulmonary vasculature. There were no changes in pulmonary artery acceleration time assessed by echocardiography (9A) or RVSP measured by invasive hemodynamics (9B) between MCT-V and GP130 antagonist rats. GP130 antagonist rats trended to having higher RVSP compared to control (p=0.068). N=9-16 animals per group. Representative images of small pulmonary arterioles from control (9C), MCT-V (9D), and GP130 antagonist (9E) rats. Scale bar 10 μm. Percent medial wall thickness quantified in (9F). N=4 rats per group, 20-31 small pulmonary arterioles measured per animal. (*) indicates statistically different from control as determined by one-way ANOVA with Tukey post-hoc analysis or Brown-Forsythe and Welch ANOVA with Dunnett post-hoc analysis. GP130 antag: glycoprotein 130 antagonist; MCTV: monocrotaline-vehicle. RVSP: right ventricular systolic pressure.

FIGS. 10A-10G: Higher IL-6 levels in PAH patients are associated with worse RV function independent of changes in the pulmonary vasculature. Higher IL-6 levels are associated with lower RV FAC (10A) and higher NT-proBNP levels (10B). There were no differences in mPAP (p=0.35) (10C), PVR (p=0.18) (10D), or PAC (p=0.32) (10E) between patients with higher IL-6 levels compared to those with lower IL-6 levels. (10F) Relationship between RV FAC and mPAP. Patients with higher IL-6 levels had lower RV FAC at each mPAP compared to patients with lower IL-6 levels (statistical difference in y-intercept, p=0.02; no difference in slope, p=0.84). (10G) Relationship between RV FAC and PVR. Patients with higher IL-6 had lower RV FAC at each PVR compared to patients with lower IL-6 (statistical difference in y-intercept, p=0.02; no difference in slope, p=0.56). N=73 total patients, with the two groups stratified by median IL-6 level. (*) indicates statistically different as determined by t-test or Mann-Whitney U test. Simple linear regression was used to evaluate whether there were any differences between the lower and higher IL-6 curves in (10F) and (10G). IL-6: interleukin-6; NT-proBNP: N-terminal pro B-type natriuretic peptide; PAC: pulmonary arterial compliance; mPAP: mean pulmonary arterial pressure; PVR: pulmonary vascular resistance; RV FAC: right ventricular fractional area change; WU: Wood units.

FIG. 11. Metabolomics data showing that SC-144 is able to restore the metabolic signature of the right ventricle in rats with PAH.

FIGS. 12A-12H: GP130 antagonism blunted RV STAT3 activation, normalized the MT-JPH2 pathway, and restored t-tubule architecture. (12A) Representative Western blots and (12B) quantification of protein abundance in RV extracts from control, MCT-V, and SC-144 rats demonstrated GP130 inhibition normalized expression of GP130, STAT3, pSTAT3, and the ratio of pSTAT3/STAT3. Data shown as expression relative to control (n=4 per group). Representative confocal micrographs of RV free wall sections showed SC-144 reduced the amount of (12C) GP130 receptors (white arrowheads) at the cell membrane and (12D) pSTAT3 (yellow arrowheads) in nuclei. (12E) Representative Western blots and (12F) protein quantification showed GP130 antagonism reduced expression of a- and b-tubulin, detyrosinated α-tubulin, and MAP4 and increased JPH2. Data shown as expression relative to control (n=4 per group). (12G) Representative confocal images of RV free wall sections stained with Alexa Fluor-633 conjugated wheat germ agglutinin. SC-144 restored RV t-tubule architecture (red arrowheads). (12H) Quantification of t-tubule architecture and organization by TTorg (arbitrary units are TTpower). n=3 animals per group, ≥7 cardiomyocytes were quantified per animal. *p<0.05 and ****p<0.0001 as assessed by Brown-Forsythe and Welch ANOVA with Dunnett post-hoc analysis.

FIGS. 13A-13B: Dysregulation of the GP130 and microtubule-JPH2 pathways were less pronounced in the LV. (13A) Representative Western blots and (13B) quantification of protein abundance in LV extracts from control, MCT-V, and SC-144.

FIGS. 14A-14J: Quantitative proteomic analysis of MAPs in the RV revealed a link between microtubule remodeling and mitochondrial dysfunction. (14A) Principal component analysis showed GP130 antagonism partially restored the global expression signature of MAPs. (14B) Hierarchical cluster analysis of MAPs demonstrated the expression pattern of MAPs in the RV of SC-144 rats more closely resembled control than MCT-V. (14C) Ten most significantly enriched pathways identified using Ingenuity pathway analysis of dysregulated MAPs in MCT-V RV when compared to control. The two most enriched pathways were mitochondrial dysfunction and oxidative phosphorylation. Hierarchical cluster analysis of proteins in complex I (14D), complex II (14E), complex III (14F), complex IV (14G), and complex V (14H), the TCA cycle (14I), and fatty acid oxidation (14J). SC-144 corrected dysregulation of nearly all mitochondrial metabolic proteins.

FIG. 15: Statistical summary and random forest analysis of the RV metabolomics results performed by Metabolon.

FIGS. 16A-16N: SC-144 improved the RV metabolic signature, restored mitochondrial morphology, and corrected mitochondrial fission/fusion imbalance. (16A) Principal component analysis demonstrated SC-144 normalized the global RV metabolic signature. Hierarchical cluster analysis of (16B) glycolysis, (16C) glutaminolysis, and (16D) acylcarnitine metabolites identified a normalization of multiple metabolic pathways with SC-144. (16E) Representative electron micrographs of mitochondria. GP130 antagonism partially corrected the RV mitochondrial morphology as assessed by (16F) variability in mitochondrial area, (16G) histogram of the distribution in mitochondrial area, and (16H) histogram of the ratio of length to width (L/W), which assesses the proportion of mitochondria that were the normal, elliptical shape. SC-144 reduced the amount of spherical mitochondria with less mitochondria that were <25th percentile of control L/W (16I) and increased the proportion of mitochondria with normal, elongated shapes as quantified by proportion of mitochondria >75th percentile of control L/W (16J). While the percentage of small mitochondria (<25th percentile of control) were similar between MCT-V and SC-144 (16K), GP130 antagonism reduced the amount of very large, swollen mitochondria as quantified in (16L). (16M) Representative Western blots and (16N) quantification of protein abundance in RV extracts from control, MCT-V, and SC-144 demonstrated GP130 inhibition did not affect MFN1 and MFN2 expression but normalized OPA1, FIS1, and DRP1 expression. *p<0.05, **p<0.01, and (ns) no statistical difference as determined by one-way ANOVA with Tukey post-hoc analysis.

FIG. 17: Integration of the proteomics and metabolomics results. There was an enrichment of differentially expressed microtubule-associated proteins and metabolites in the TCA cycle, purine metabolism, pyruvate metabolism, glycolysis or gluconeogenesis, and fatty acid degradation.

FIGS. 18A-18B: Chemical stabilization of microtubules with paclitaxel in H9c2 cardiomyocytes altered mitochondrial metabolic function. (18A) Seahorse analysis of the oxygen consumption rate profile and (18B) individual parameters of mitochondrial respiration. Con: control; OCR: oxygen consumption rate; Pac: paclitaxel. *p<0.05, **p<0.01, ***p<0.001, and ****p<0.0001 as determined by t-test or Mann-Whitney U test.

FIGS. 19A-19E: GP130 antagonism did not alter pulmonary vascular disease severity. SC-144 did not change RV afterload as assessed by (19A) pulmonary artery acceleration time, (19B) RVSP, and (19C) effective arterial elastance (Ea). n=8-16 rats per group. (19D) No difference in percent medial wall thickness of small pulmonary arterioles was observed between MCT-V and SC-144. (19E) Representative images of small pulmonary arterioles. n=4 lungs per group, with ≥20 small pulmonary arterioles per animal. *p<0.05, **p<0.01, ****p<0.0001, and (ns) no statistical difference as determined by one-way ANOVA with Tukey post-hoc analysis for pulmonary artery acceleration time or Brown-Forsythe and Welch ANOVA with Dunnett post-hoc analysis for RVSP, Ea, and percent medial wall thickness.

FIGS. 20A-20H: GP130 antagonism reduced RV hypertrophy and fibrosis. SC-144 reduced RV hypertrophy as assessed by the Fulton index (RV/LV+S) (20A) and RV weight normalized to body weight (20B). n=10-26 animals per group. (20C) SC-144 decreased cardiomyocyte area. (20D) Representative images of cardiomyocytes in H&E stained RV free wall sections. n=2-3 RV per group, at least 21 cardiomyocytes measured per animal. (20E) Representative Western blots and (20F) quantification of collagen I and III protein abundance in RV extracts (n=4 per group). SC-144 decreased expression of collagen I and III compared to MCT-V, which corresponded with less RV fibrosis observed in trichrome staining of RV free wall as quantified in (20G). n=2-3 RV per group, 3-4 areas sampled per animal. (20H) Representative trichrome stained RV sections. Black arrows highlight RV fibrosis. *p<0.05, **p<0.01, ***p<0.001, ****p<0.0001, and (ns) no statistical difference as determined by one-way ANOVA with Tukey post-hoc analysis for Fulton index, RV/BW, and percent RV fibrosis or Brown-Forsythe and Welch ANOVA with Dunnett post-hoc analysis for cardiomyocyte area.

FIG. 21A-21H: SC-144 improved RV function. (21A) TAPSE, (21B) percent RV free wall thickness change, (21C) stroke volume, (21D) cardiac output, and (21E) cardiac output normalized to body weight were measured by echocardiography. n=9-14 rats per group. SC-144 improved RV function in all echocardiographic measures. Invasive hemodynamics and pressure-volume (PV) loops demonstrated that SC-144 augmented (21F) RVEF, (21G) end-systolic elastance (Ees), and (21H) RV-PA coupling (Ees/Ea). n=8-16 rats per group. *p<0.05, **p<0.01, ***p<0.001, ****p<0.0001, and (ns) no statistical difference as determined by one-way ANOVA with Tukey post-hoc analysis for TAPSE, RV free wall thickness change, stroke volume, cardiac output, cardiac output/body weight, and RVEF or Brown-Forsythe and Welch ANOVA with Dunnett post-hoc analysis for Ees and Ees/Ea.

FIG. 22: Survival curve demonstrating MCT-V rats had a 30% mortality rate during the study period. **p<0.01 vs. control and SC-144 as assessed by the log-rank test.

FIG. 23A-23G: Higher IL-6 levels in PAH patients were associated with worse RV function independent of changes in PAH severity. Higher IL-6 levels are associated with higher NT-proBNP levels (23A) and lower RVFAC (23B). (23C) Relationship between RVFAC and mPAP. Patients with higher IL-6 levels had lower RVFAC at each mPAP compared to patients with lower IL-6 levels (statistical difference in y-intercept, p=0.02; no difference in slope, p=0.84). (23D) Relationship between RVFAC and PVR. PAH patients with higher IL-6 had reduced RVFAC at each PVR compared to patients with lower IL-6 (statistical difference in y-intercept, p=0.02; no difference in slope, p=0.56). n=73 total patients with the two groups stratified by median IL-6 level. There were no differences in (23E) mPAP (p=0.35), (23F) PVR (p=0.18), or (23G) PAC (p=0.32) between patients with higher IL-6 levels compared to those with lower IL-6 levels. **p<0.01 as determined by t-test for RVFAC or Mann-Whitney U test for NT-proBNP. Linear regression evaluated differences between the lower and higher IL-6 curves in (C) and (D).

FIGS. 24A-24D: Higher IL-6 levels in PAH were associated with worse RV function when the cohort was separated into tertiles. (24A) PAH patients with the highest IL-6 levels had significantly greater NT-proBNP levels compared to the lowest IL-6 tertiles. Patients with intermediate IL-6 levels have intermediate NT-proBNP levels but significantly higher than the lowest tertile. (24B) RVFAC is decreased in PAH patients with the highest IL-6 levels compared to the lowest IL-6 tertile. Patients with the middle IL-6 levels trended towards having intermediate RVFAC. (24C) Relationship between RVFAC and mPAP. Patients with higher IL-6 levels had lower RVFAC at every mPAP (statistical difference in y-intercept, p=0.02; high IL-6 y-intercept: 28.7%, medium IL-6 y-intercept: 32.1%, low IL-6 y-intercept: 41.2%; no difference in slope, p=0.9). (24D) Relationship between RVFAC and PVR. Patients with higher IL-6 levels had decreased RVFAC at most PVR levels (statistical difference in y-intercept, p=0.03; high IL-6 y-intercept: 26.3%, medium IL-6 y-intercept: 28.1%, low IL-6 y-intercept: 38.6%; no statistical difference in slope, p=0.52).

FIG. 25: GP130-mediated RV dysfunction in PAH. Heightened GP130 activation induces STAT3-mediated gene transcription of microtubule proteins. This causes microtubule remodeling, which leads to JPH2 dysregulation, t-tubule derangements, and mitochondrial dysfunction via imbalance of mitochondrial fission/fusion. These molecular changes ultimately manifest as RV dysfunction.

FIG. 26: Transcript expression of IL-6, GP130, STAT3, and tubulins in human LV and RV cardiomyocytes. Data obtained from the Human Cell Atlas⁴⁴, which has single-cell and single-nucleus RNA sequencing data from donor hearts. There were five ventricular cardiomyocyte populations (vCM1-5) identified in the study⁴⁴. The RV has a higher proportion of group 2 cardiomyocytes (vCM2_RV) than the LV (39.91 vs. 9.12%), suggesting that this population of cardiomyocytes may have an important role of RV function. Genes that are expressed higher in RV than LV group 2 cardiomyocytes are bolded.

### DETAILED DESCRIPTION

Pulmonary Arterial Hypertension (PAH)

Pulmonary arterial hypertension (PAH) is a progressive disease caused by changes in multiple cell types in the pulmonary vasculature. There is endothelial cell dysfunction with exaggerated secretion of vasoconstrictive, pro-proliferative substances, such as endothelin, and impaired release of vasodilatory, anti-proliferative molecules, such as nitric oxide and prostacyclin. This imbalance increases pulmonary artery smooth muscle cell (PASMC) tone and proliferation rates. In PASMCs, there is hypercontractility, proliferation and apoptosis resistance due to genetically and epigenetically controlled mechanisms, calcium mishandling, metabolic reprogramming, and disrupted mitochondrial dynamics. Extracellular matrix remodeling promotes PAH by increasing vessel stiffness which activates signaling pathways that induce metabolic derangements via mechano-transduction. Finally, there is a marked inflammatory response with T-cell, B-cell, and dendritic cell infiltration into the pulmonary vasculature and elevated levels of circulating inflammatory cytokines. The summation of these molecular, cellular, and histological changes manifests as reduced pulmonary arterial compliance (PAC) and elevated pulmonary vascular resistance (PVR) and pulmonary arterial pressures which, in aggregate, augment the workload of the right ventricle (RV).

The RV in PAH

As the pulmonary vascular remodeling in PAH progresses, the systolic pressure overload on the RV leads to RV hypertrophy, fibrosis, and metabolic derangements that cause RV failure. RV dysfunction (RVD) is the strongest predictor of mortality in PAH and RVD is the reason that the median survival in PAH is only 5-7 years. Although RVD is the main cause of death in PAH, no PAH therapies actually target the RV directly. This may be due to the fact that the pathophysiological mechanisms that mediate RVD are incompletely understood.

Perhaps the most rigorously characterized cellular phenotype of RVD in PAH is metabolic remodeling. In RV pressure overload, cardiomyocytes exhibit mitochondrial dysfunction which is accompanied by induction of anaerobic glycolysis and glutaminolysis, a pathological metabolic process that utilizes glutamine to regenerate Kreb cycle intermediates. Activation of mitochondria using a small molecule that stimulates pyruvate dehydrogenase, which increases pyruvate utilization by the mitochondria and enhances oxidative capacity, improves RV function in rodent PAH. Furthermore, inhibition of glutaminolysis augments mitochondrial function and blunts RVD in vivo. Thus, there is substantial evidence that metabolic derangements promote RVD in PAH, and importantly inhibiting these pathologic metabolic changes enhances RV function. However, the relationship between metabolic abnormalities and other defined cellular phenotypes associated with RVD are less well understood.

The Microtubule (MT)-Junctophilin-2 (JPH2) Pathway in RVD

It was recently shown that pathological MT remodeling causes dysregulation of JPH2, t-tubule remodeling, and RVD in the monocrotaline (MCT) rat model of PAH. Colchicine-induced MT depolymerization increases JPH2 expression and corrects t-tubule architecture, which improves RV function and enhances exercise capacity. Moreover, an independent study demonstrated JPH2 is downregulated, there is t-tubule remodeling, and RVD in MCT rats. Moreover, this group showed the degree of RVD is proportional to the JPH2 downregulation. Collectively, these data demonstrate a crucial role for the MT-JPH2 pathway in RVD in PAH. However, the upstream signaling cascade that promotes MT polymerization and JPH2 dysregulation and the other key interacting proteins in this pathway have not yet been defined.

Interleukin-6 (IL6) in PAH and Cardiac Dysfunction

IL6 is an inflammatory cytokine that binds to the cell membrane proteins IL6 receptor and GP130, which activates the downstream signaling molecule signal transducer and activator of transcription 3 (STAT3). STAT3 is normally located in the cytoplasm, but activation of the IL6 pathway results in STAT3 phosphorylation. Phosphorylated STAT3 (pSTAT3) then dimerizes and enters the nucleus where it can function as a transcription factor.

Serum levels of IL6 are elevated in PAH patients, and higher levels of IL6 are associated with increased mortality. Interestingly, IL6 levels are not correlated with markers of pulmonary vascular disease in PAH patients. However, it was recently shown that serum IL6 levels exhibit a negative logarithmic relationship with RV function in PAH. PAH patients with higher serum IL6 levels have worse RV function despite having similar pulmonary vascular disease burden when compared to patients with lower IL6 levels, suggesting IL6 can promote RVD. However, a molecular mechanism for this clinical observation is lacking.

Preclinical studies demonstrate IL6 signaling promotes cardiac dysfunction directly. First, expression of a dominate-negative GP130 blunts STAT3 activation and protects mice from left ventricular (LV) remodeling in response to transverse aortic constriction (TAC). Second, mice overexpressing a GP130 transgene that excessively activates STAT3 have lower LV fractional shortening and higher mortality after myocardial infarction. Finally, IL6 knockout mice exhibit blunted STAT3 activation, less pathological LV remodeling, and they are able to maintain cardiac function in response to TAC better than control mice. Collectively, these data demonstrate that IL6 signaling via STAT3 promotes pathological LV remodeling, but the effects of IL6 on the RV in PAH are unexplored.

STAT3 Induces Microtubule Remodeling

Previous studies demonstrated STAT3 can directly regulate the microtubule cytoskeleton. First, STAT3 overexpression in fibroblasts promotes microtubule stabilization by binding and sequestering the microtubule depolymerizing protein stathmin. Second, in T-cells, small molecule-based inhibition of STAT3 phosphorylation alters microtubule organization and reduces the abundance of stabilized microtubules. A third study showed STAT3 directly binds and fortifies microtubules. Finally, activation of STAT3 via IL6 induces microtubule stabilization in neonatal cardiomyocytes. Clearly, there is substantial evidence that STAT3 regulates the microtubule cytoskeleton, with multiple proposed mechanisms.

It has been shown that STAT3 and phosphorylated STAT3 (pSTAT3) levels are elevated in the RV of MCT and pulmonary artery banded (PAB) rats, and higher levels are associated with more severe RVD. Moreover, pSTAT3 is positively correlated with tubulin levels in RV extracts. In isolated cardiomyocytes, IL6 induces MT remodeling, blunts the JPH2 enrichment at TT, and causes TT derangements.

Methods of Treatment

In certain embodiments, the present invention provides a method of treating right ventricular dysfunction (RVD) in pulmonary hypertension by administering a GP130 antagonist in a patient in need thereof. As used herein, right ventricular dysfunction is defined as chronic dysfunction of the right ventricle leading to reduced cardiac output (CO). RVF occurs when the RV fails to provide enough blood flow to the pulmonary circulation to accomplish adequate left ventricle filling with ultimately results in end-organ hypoperfusion with the consequent multiorgan dysfunction/failure. In certain embodiments, the pulmonary hypertension is pulmonary arterial hypertension (PAH), pulmonary hypertension owing to left heart disease, pulmonary hypertension due to chronic lung disease, chronic thromboembolic pulmonary hypertension (CTEPH), or pulmonary hypertension with unclear multifactorial mechanisms.

In certain embodiments, treating PH improves right ventricular function by 10% to 40% as compared to pre-treatment function. In certain embodiments, the right ventricular function is improved by 10%, 20%, 30%, or 40%, as compared to pre-treatment function.

In certain embodiments, the present invention provides a method of treating a patient having an elevated blood level of IL-6 (>3.0 pg/mL) by administering an in a patient in need thereof.

The terms “treat,” “treatment,” or “treating” to the extent it relates to a disease or condition includes inhibiting the disease or condition, eliminating the disease or condition, and/or relieving one or more symptoms of the disease or condition. The terms “treat,” “treatment,” or “treating” also refer to both therapeutic treatment and/or prophylactic treatment or preventative measures, wherein the object is to prevent or slow down (lessen) an undesired physiological change or disorder, such as, for example, the development or spread of right ventricular dysfunction. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat,” “treatment,” or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat,” “treatment,” or “treating” does not include preventing or prevention.

In certain embodiments, the administration is by oral administration.

In certain embodiments, the administration is by intravenous infusion.

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

Therapeutic Agents

In certain embodiments, the IL-6 antagonist targets an IL-6 co-receptor. As used herein, “targets” means that the IL-6 antagonist inactivates the biological function of an IL-6 co-receptor.

In certain embodiments, the IL-6 co-receptor is Glycoprotein 130 (GP130). GP130 (also known as gp130, IL6ST, IL6-beta or CD130) is a transmembrane protein which is the founding member of the IL-6 superfamily. It forms one subunit of the type I cytokine receptor within the IL-6 receptor family.

In certain embodiments, the IL-6 antagonist is SC-144 (2-(7-Fluoropyrrolo[1,2-a]quinoxalin-4-yl) 2-pyrazinecarboxylic acid hydrazide hydrochloride).

The pharmaceutical compositions of the invention can comprise one or more excipients. When used in combination with the pharmaceutical compositions of the invention the term “excipients” refers generally to an additional ingredient that is combined with the IL-6 antagonist to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

The IL-6 antagonist can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the IL-6 antagonist can be determined by comparing their in vitro activity, and in vivo activity in animal models. Methods for the extrapolation of effective dosages in mice, and other animals, to humans are known to the art; for example, see U.S. Pat. No. 4,938,949.

The amount of the IL-6 antagonist required for use in treatment will vary with the route of administration, and the age and condition of the patient, and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

Compounds of the invention can also be administered in combination with other therapeutic agents. Examples of such agents include typical PH treatment such as phosphodiesterase inhibitors, endothelin receptor antagonist, soluble guanylate cyclase activators, IP3 receptor agonist, and prostacyclins. Accordingly, in one embodiment the invention also provides a composition comprising a GP130 antagonist, at least one other therapeutic agent, and a pharmaceutically acceptable diluent or carrier. The invention also provides a kit comprising a GP130 antagonist, at least one other therapeutic agent, packaging material, and instructions for administering the IL-6 antagonist and the other therapeutic agent or agents to an animal to treat right ventricular dysfunction.

In certain embodiments, the first and second compositions are administered simultaneously. For example, a first composition comprising a GP130 antagonist is administered along with a typical PH treatment. In certain embodiments, the first and second compositions are administered sequentially. For example, a first composition comprising a GP130 antagonist is administered followed by administration with a typical PH treatment, or a typical PH treatment is administered followed by administration of a GP130 antagonist. In certain embodiments, the first and second compositions are administered separately.

Certain embodiments of the invention will now be illustrated by the following non-limiting Examples.

### Example 1

### Rodent Models of RV Pressure Overload

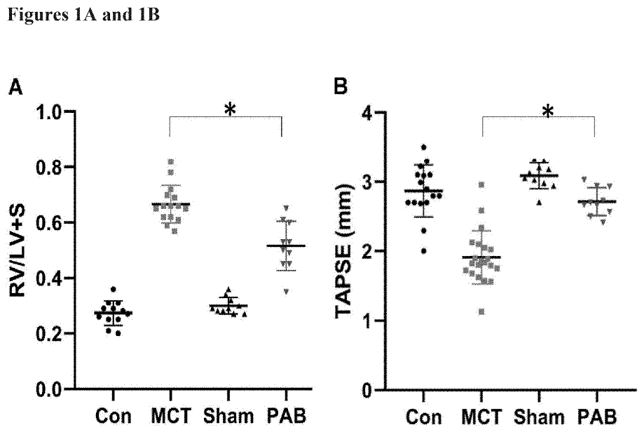

The RV phenotype of two rat models of RV pressure overload was characterized. Echocardiographic analysis revealed pulmonary artery banded (PAB) rats exhibited right ventricular hypertrophy (RVH) (FIG. 1A) but relatively preserved RV function with only minimally reduced tricuspid annular plane systolic excursion (TAPSE), an echocardiographic measure of RV function (FIG. 1B). Conversely, monocrotaline (MCT) induced-PAH rats had more RVH (FIG. 1A) and a TAPSE value that was significantly lower than PAB rats (FIG. 1B). Thus, these two rat models exhibit distinct RV phenotypes that are useful for the examples herein.

IL6 Signaling is Associated with MT-JPH2 Pathway and RVD Severity in RV Pressure Overload

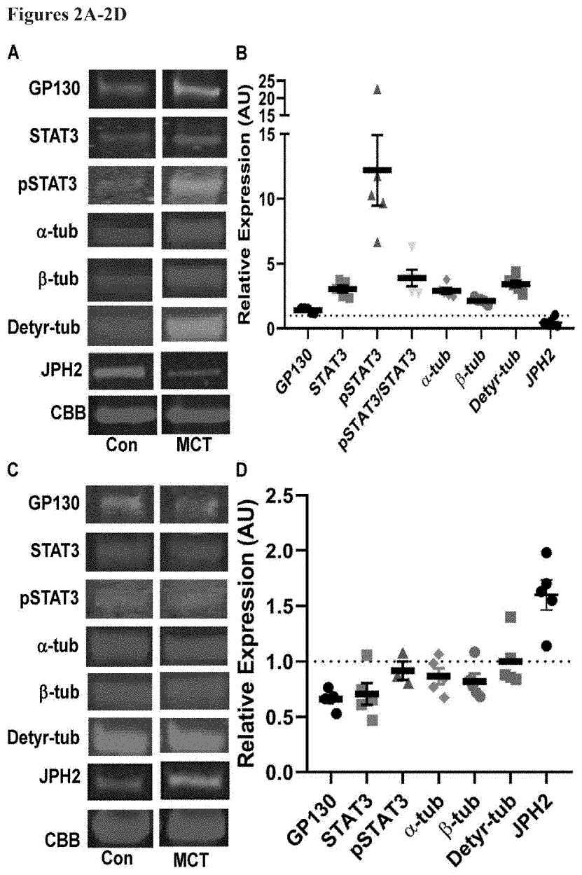

Next, the relationship between IL6 signaling, the MT-JPH2 pathway, and RVD severity in MCT and PAB rats was examined. In MCT rats, there was an induction of IL6 signaling with increased abundance of GP130 (1.4±0.1 fold increase), total STAT3 (3.1±0.3 fold increase), and pSTAT3 (12.2±2.7 fold increase). This was associated with elevated levels of α-(2.9±0.2 fold increase) and β-tubulin (2.2±0.1 fold increase), the individual subunits of MT. Moreover, levels of detyrosinated α-tubulin (3.5±0.3 fold increase), a marker of stabilized MT, were higher. Finally, JPH2 levels were reduced in the MCT RV (0.4±0.1 relative abundance) (FIGS. 2A and 2B). In the unaffected left ventricle (LV) of MCT rats, IL6 signaling was not enhanced, and that was associated with less MT stabilization and higher levels of JPH2. GP130 (0.7±0.04 relative abundance), total STAT3 (0.7±0.1 relative abundance), and pSTAT3 (0.9±0.1 relative abundance) were either decreased or unchanged. α-(0.9±0.1 relative abundance) and β-tubulin (0.8±0.1 relative abundance) were slightly lower and detyrosinated α-tubulin (1.0±0.1 relative abundance) levels were unchanged. Finally, there was an increase in JPH2 levels (1.6±0.1 fold increase) (FIGS. 2C and 2D). In summary, these data demonstrate IL6 signaling is associated with activation of the MT-JPH2 pathway in RV pressure overload. Importantly, the lack of IL6 activation in the unaffected LV suggests these findings are specific to the diseased ventricle and they are not simply due to systemic inflammation.

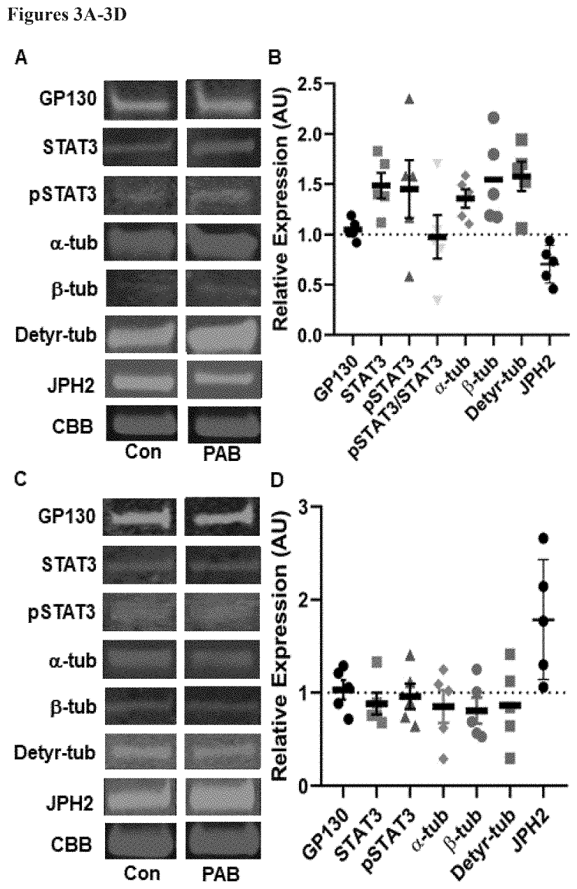

In PAB rats, which exhibit less RVD than MCT rats (FIGS. 1A-1B), IL6 signaling was not as strongly induced as GP130 (1.0±0.1 fold increase) levels were not changed, and STAT3 (1.5±0.1 fold increase) and pSTAT3 (1.5±0.3 fold increase) were much less dramatically elevated. This was associated with less upregulation of α-tubulin (1.4±0.1 fold increase), β-tubulin (1.5±0.2 fold increase), and detyrosinated α-tubulin levels (1.6±0.2 fold increase). Moreover, JPH2 levels were not as markedly reduced (0.7±0.1 relative abundance) (FIGS. 3A and 3B). We also examined the IL6-MT-JPH2 relationship in the unaffected LV of PAB rats. GP130 (1.0±0.1 fold increase), STAT3 (0.9±0.1 fold increase) and pSTAT3 (1.0±0.1 fold increase) levels were unchanged (FIGS. 3C and 3D). There were minimal changes in α-(0.9±0.1 fold increase), β-(0.8±0.2 fold increase), and detyrosinated (0.9±0.2 fold increase) tubulin levels (FIGS. 3C and 3D). Finally, JPH2 levels were elevated (1.6±0.5 fold increase) (FIGS. 3C and 3D). In conclusion, the results from the MCT and PAB rats established a relationship between IL6 signaling and the MT-JPH2 pathway and revealed that RVD severity is proportional to these newly linked pathways.

IL6 Directly Activates the MT-JPH2 Pathway

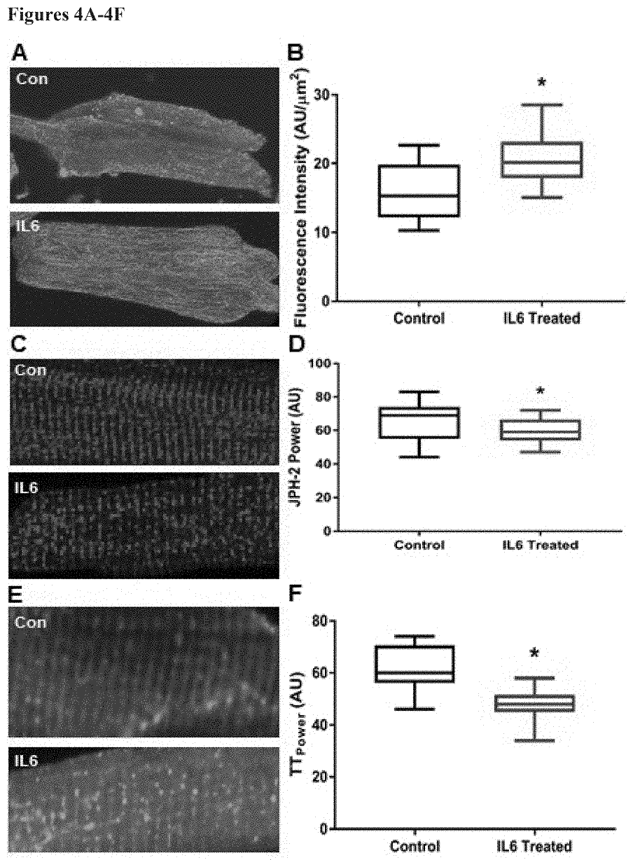

To examine the direct effects of IL6 on the MT-JPH2 pathway, adult cardiomyocytes were incubated with IL6 (10 ng/mL) and then it was quantified how MT, JPH2, and t-tubules were affected. IL6 increased MT density as determined by quantitative immunofluorescence (16.0±0.9 vs 20.4±0.7 fluorescence intensity/μm², p<0.001) (FIGS. 4A and 4B). The altered MT structure disrupted JPH2 localization marked by loss of t-tubule enrichment (TT score: 65.6±2.5 vs 59.6±1.6, p=0.04) (FIGS. 4C and 4D). Finally, IL6 caused t-tubule remodeling with loss of t-tubule regularity (TT score: 59.9±1.9 vs 51.9±1.5, p=0.003) (FIGS. 4E and 4F). In summary, these data demonstrated that IL6 directly caused MT-induced JPH2 mislocalization and t-tubule derangements in mature cardiomyocytes.

### Example 2

### IL6 Signaling Promotes Microtubule Stabilization, JPH2 Dysregulation, and RVD, and that Inhibition of IL6 Signaling Mitigates RVD In Vivo

Examination of the IL6 Signaling Pathway and Relative Activation of the MT-JPH2 Pathway in Rodent Models of Pressure Overload.

Animal Models: The MCT and PAB rat models of RV pressure overload are used. For the MCT model, Sprague-Dawley rats are subcutaneously injected with 60 mg/kg of monocrotaline (Sigma). For PAB rats, adult Sprague-Dawley rats are be anesthetized with 3% isoflurane and intubated. The main pulmonary artery (PA) is carefully dissected from the ascending aorta via a limited median sternotomy. A 1.3-mm diameter needle is placed parallel to the main PA and ligated with a 4-0 silk suture. The needle is then withdrawn to create a fixed PA stenosis. Age- and sex-matched control rats undergo a sham surgery.

Quantitative RT-PCR: Total RNA is purified from the RV and LV using the RNAzol kit (Sigma). Specific primers for IL6 receptor, GP130, STAT3, JPH2, all eight α-tubulin isoforms, and all eight β-tubulin isoforms are used to quantify mRNA levels using the QuantStudio 6 Flex system (Applied Biosciences).

Western blot analysis: 100 mg of frozen RV and LV is extracted in sodium dodecyl sulfate buffer. Quantitative western blot analysis is performed using the LiCor infrared imaging system. Levels of IL6 receptor, GP130, STAT3, phosphorylated STAT3, α-tubulin, β-tubulin, and detyrosinated α-tubulin protein are measured to quantify IL6 signaling in both the right and left ventricles in MCT and PAB rats with age- and sex-matched rats serving as controls.

Confocal microscopy: 10 μm cryosections are collected, fixed in ice-cold acetone or methanol and processed for immunofluroescence. Confocal micrographs of sections stained with primary antibodies to IL6 receptor, GP130, STAT3, phosphorylated STAT3, α-tubulin, β-tubulin, and detyrosinated α-tubulin are collected on a super resolution Nikon A1RSi microscope.

Evaluation Whether IL6 Directly Activates the MT-JPH2 Pathway that Causes t-Tubule Remodeling, Calcium Mishandling, and Contractile Dysfunction in Isolated Cardiomyocytes.

Determination of how IL6 alters the microtubule-JPH2-t-tubule pathway in isolated cardiomyocytes: Right ventricular cardiomyocytes are isolated using the Langendorf retrograde perfusion protocol. Cardiomyocytes are incubated for 24, 48, and 72 hours with either phosphate buffered saline (PBS), IL6 (10 ng/mL), IL6 (10 ng/mL) plus the STAT3 inhibitor Stattic (2 μM), or IL6 (10 ng/mL) plus colchicine (10 μM). Then Western blot analysis and confocal microscopy is used to examine microtubule density, JPH2 protein levels and localization, and t-tubule morphology. JPH2 localization patterns and t-tubule remodeling are quantified using the TT_{Power} plug-in in FIJI.

Examination of how IL6 affects calcium handling in isolated cardiomyocytes: Cardiomyocytes are cultured for 24, 48, and 72 hours with either PBS, IL6 (10 ng/mL), IL6 (10 ng/mL) plus Stattic (2 μM), or IL6 (10 ng/mL) plus colchicine (10 μM). For Ca²⁺ measurements, myocytes are first incubated in the dark at room temperature with 2 μM Fura (Thermo Fisher Scientific) for 10 min, followed by de-esterification for an additional 20 min. Myocytes are bathed in Tyrode's solution at 30° C. and visualized on an inverted Nikon Eclipse TE2000-U microscope. Myocytes are stimulated at 1.0 Hz and 25V, and data are acquired at 1000 Hz. Traces are averaged and analyzed with IonWizard software (IonOptix).

Determination Whether IL6 Antagonism Improves RV Function In Vivo by Inhibiting MT-Induced JPH2 Dysregulation and t-Tubule Remodeling

Animal models and treatment: For the GP130 antagonists treatment experiments there is a control group, a RV pressure overload treated with the GP130 inhibitor SC144 (Sigma-Aldrich) and a RV pressure overload treated with vehicle group. For the MCT rats, PAH is allowed to develop for two weeks and then rats are treated SC144. Four weeks after MCT injection, end-point analysis is conducted. For PAB rats, the rats are allowed to recover from surgery and develop RVD for four weeks. Then, rats are treated at week four with either SC144 or vehicle as outlined above with end-point analysis being performed eight weeks after PAB.

Western blot analysis: Western blot analysis is carried out as described above.

Examination of t-tubule structure: T-tubule structure is examined with confocal and electron microscopy (EM). Fluorescent labeled wheat-germ agglutinin delineates t-tubules in cryosections and in isolated cardiomyocytes from the LV and RV in all rat models. Confocal images are collected and t-tubule regularity is quantified using the TT_{power} plug-in for FIJIμ.

Echocardiographic assessment of right ventricular function: Echocardiography is performed using a Vevo2100 ultrasound system with a 37.5-MHz transducer (Visual Sonics, Inc). Rats are lightly anesthetized with isoflurane (1.6% to 2.0%), and chest hair is removed using a depilatory cream (Nair®). M-mode and 2-D modalities are used to measure RV free wall (RVFW) thickness during end diastole and end systole and tricuspid annular plane systolic excursion (TAPSE). PA diameter is measured at the level of the pulmonary outflow tract during mid-systole. Pulsed-wave Doppler is used to measure PA acceleration time and PA flow velocity time integral. RV ejection time is measured as the interval from the onset to the end of ejection in milliseconds. Stroke volume, cardiac output, and cardiac index is calculated as previously described.

Hemodynamic Evaluation: A high-fidelity catheter (Scisence pressure-volume catheter; Transonic) is advanced into the RV via the jugular vein and right atria, in closed-chest rats. RV pressure and volume are recorded continuously using the Pressure-Volume Measurement System (Transonic). Heart rate, right ventricular systolic pressure, cardiac output, ejection fraction, stroke volume, and dp/dt are recorded.

### Example 3

### GP130 Signaling Promotes Right Ventricular Dysfunction in Pulmonary Arterial Hypertension

Rationale: Pulmonary arterial hypertension (PAH) is a pulmonary arterial vasculopathy that ultimately results in right ventricle (RV) dysfunction (RVD) and death. It was previously shown that microtubule-mediated junctophilin-2 (MT-JPH2 pathway) dysregulation causes t-tubule disruption and RVD. However, the upstream regulators of this pathway are unknown. Previous studies show signaling via glycoprotein 130 (GP130) promotes microtubule remodeling in cultured cardiomyocytes, but the effects of GP130 signaling on the RV in PAH are unexplored.

Objective: Investigate if GP130 signaling via signal transducer and activator of transcription 3 (STAT3) regulates the MT-JPH2 pathway and if inhibition of GP130 modulates RVD in preclinical PAH. In human PAH, examine the relationship between the GP130 ligand, interleukin-6 (IL-6) and RV function.

Methods and Results: Immunoblots of RV extracts demonstrated that GP130 antagonism blunted STAT3 activation, which normalized the MT-JPH2 pathway in the RV of MCT rats. Restoration of the MT-JPH2 pathway reversed pathological t-tubule remodeling leading to enhanced RV function, as determine by echocardiography and invasive hemodynamic studies, and improved survival rates. Moreover, GP130 antagonism blunted RV hypertrophy, decreased expression of collagen I and III, and reduced RV fibrosis. Quantitative proteomics analysis of the RV microtubule-associated protein (MAP) fraction showed GP130 antagonism normalized the expression patterns of >2800 MAPs. Pathway analysis revealed enrichments of mitochondrial and metabolic pathways, suggesting a link between microtubule remodeling and metabolic derangements exists. Importantly, the improvements in the RV molecular signature, structure, and function with GP130 antagonism occurred despite no differences in pulmonary vascular disease severity. Finally, in 73 PAH patients, elevated serum levels of IL-6 were associated with more severe RVD despite similar hemodynamic severity of PAH.

Conclusions: Inhibiting GP130 signaling enhances RV function, via modulation of the microtubule cytoskeleton, independent of changes in the pulmonary vasculature. Thus, targeting GP130 has therapeutic value for RV dysfunction in PAH.

### INTRODUCTION

Pulmonary arterial hypertension (PAH) is a lethal pulmonary arterial vasculopathy that increases right ventricular (RV) afterload and results in RV dysfunction (RVD). RVD is the strongest predictor of mortality in PAH1-5, but the molecular mechanisms that mediate RVD are incompletely understood. This is highlighted by the fact that current therapies for PAH are directed at the pulmonary vasculature and none directly target the RV. Additionally, RVD is not solely due to increased afterload as some PAH patients have progressively worsening RVD despite treatment with pulmonary vasodilators and reduction in afterload. This suggests there are non-hemodynamic regulators of RV function that could be modulated to enhance RV function.

It was previously shown that microtubule-mediated junctophilin-2 (MT-JPH2 pathway) downregulation in RV cardiomyocytes promotes t-tubule disruption and RVD in PAH8. However, the upstream mediators of the MT-JPH2 pathway are unknown. Interestingly, a previous study showed that signaling through the cytokine receptor, glycoprotein-130 (GP130) via signal transducer and activator of transcription 3 (STAT3) increased tubulin content and stabilized microtubles in neonatal cardiomyocytes. This is directly relevant to PAH as elevated levels of the GP130 ligand, interleukin-6 (IL-6) are associated with worse survival rates despite minimal differences in hemodynamics. Thus, GP130 signaling may modulate the MT-JPH2 pathway to promote RVD, which may explain why elevated levels of IL-6 are associated with worse survival in PAH.

The objective of the present study was to investigate whether there is increased GP130 signaling in the RV of the monocrotaline (MCT) rat model of PAH, and determine whether inhibition of GP130 signaling and STAT3 activation would reverse MT-mediated JPH2 activation, t-tubule disruption, and RVD in MCT rats. Finally, the relationship between IL-6 levels and RV function in PAH was examined to determine if this relationship is important in human PAH.

Methods

Animal models: Male Sprague Dawley rats (˜200-250 g; ˜7-8 weeks old) (Charles River Laboratories, Wilmington, Mass.) received a single subcutaneous injection of monocrotaline (MCT) (60 mg/kg) (Sigma-Aldrich, St. Louis, Mo.) or phosphate-buffered saline (control). Two weeks after MCT injection, rats received daily intraperitoneal injections of a GP130 antagonist, SC-144 (10 mg/kg) (ApexBio, Houston, Tex.), or vehicle for 10 days and endpoint analysis was completed the same day that the animals received their last injection of GP130 antagonist or vehicle. SC-144 was dissolved in dimethyl sulfoxide (DMSO) to a concentration of 200 mg/mL, diluted to 20 mg/mL in propylene glycol, and further added to 0.9% NaCl and 40% propylene glycol to achieve a final concentration of 10 mg/kg. Animal studies were approved by the University of Minnesota Institutional Animal Care and Use Committee.

Western blot analysis: Immunoblots with 25 μg of RV protein extracts were performed using the Odyssey Infrared Imaging system (Lincoln, Nebr.). Post transfer SDS-PAGE gels were stained with Coomassie brilliant blue and imaged at the 700-nm wavelength on the Odyssey Imaging System with the band corresponding to the myosin heavy chain used as the loading control. Antibodies used in this study are listed in Table 1.

TABLE 1
Antibodies Used in Study.

| | | | Catalogue | |
|---|---|---|---|---|
| Antigen | Company | Species | Number | Dilution |
| α-tubulin | Millipore | Mouse | 05-829 | 1:250 |
| β-tubulin | Sigma-Aldrich | Mouse | T4026 | 1:250 |
| Detyrosinated α- | Abcam | Rabbit | Ab48389 | 1:250 |
| tubulin | | | | |
| Junctophilin-2 | Invitrogen | Rabbit | 405300 | 1:250 |
| GP130 | Cell Signaling | Rabbit | 3732 | 1:250 |
| | Technology | | | |
| Stat3 | Cell Signaling | Mouse | 9131 | 1:250 |
| | Technology | | | |
| P-Stat3 (Y705) | Abcam | Rabbit | Ab76316 | 1:250 |
| Collagen I | Abcam | Rabbit | Ab34710 | 1:250 |
| Collagen III | Abcam | Rabbit | Ab6310 | 1:100 |
| Anti-mouse | Li-COR | Goat | 926-32210 | 1:5000 |
| secondary | | | 926-68070 | |
| Anti-rabbit secondary | Li-COR | Goat | 926-32211 | 1:5000 |
| | | | 926-68071 | |

Rodent echocardiography: Echocardiography was performed using a Vevo2100 ultrasound system with a 37.5-MHz transducer (VisualSonics). M-mode and 2-D modalities were used to measure tricuspid annular plane systolic excursion (TAPSE) and RV free wall (RVFW) thickness during end diastole and end systole. Pulmonary artery (PA) diameter was measured at the level of the pulmonary outflow tract during mid-systole. Pulsed-wave Doppler was used to measure PA flow velocity time integral. Cardiac output was calculated.

Hemodynamic studies: Invasive closed-chest right heart catheterization was performed to obtain RV pressure-volume (PV) loops. Rats were anesthetized initially with 5% isoflurane induction and then maintained on 2-3% isoflurane. During the catheterization, rats were ventilated. A high-fidelity catheter (Scisense 1.9F pressure-volume, Transonic Systems, Ithaca, N.Y.) was advanced into the RV via the right internal jugular vein and right atria in closed-chest rats. RV pressure and volume were continuously recorded using Transonic AV500 Pressure-Volume Measurement System and Lab Scribe version 4 (iWorx Systems, Dover, N.H.).

Cardiac histology: For the t-tubule analyses, 10-μm RV free wall cryosections were fixed in 4% paraformaldehyde, washed twice with PBS for 5 minutes, and incubated with Alexa Fluor-488 conjugated wheat germ agglutinin (Sigma-Aldrich, St. Louis, Mo.) for 10 minutes. Sections were mounted in Anti-Fade Reagent (Molecular Probes, Eugene, Oreg.). Images were obtained using a Bio-Rad MRC 1000 (Hercules, Calif.) scan head mounted on an upright Nikon Optishot microscope (Tokyo, Japan) at a magnification of 1000× with immersion oil at the University of Minnesota Imaging Center. Z-stack images were converted into a Z-project using ImageJ (National Institutes of Health, Bethesda, Md.), and t-tubule structure and regularity were quantified with the TTorg plugin on ImageJ.

For the cardiomyocyte and fibrosis analyses, 10-μm RV cryosections were stained with hematoxylin and eosin (H&E) (catalog number H-3502, Vector Laboratories, Burlingame, Calif.) or trichrome stain kits (catalog number ab150686, Abcam, Cambridge, Mass.) per manufacturer's protocol, respectively. Images were obtained on a Zeiss AxioCam IC (Oberkochen, Germany) at 200× magnification. Cardiomyocyte area and percent fibrosis (determined by measuring the area of tissue stained blue divided by total tissue area) were assessed with ImageJ,

Lung histology: Lung sections were fixed in 10% formalin, embedded in paraffin, sectioned at 4-μm, and stained with H&E by the University of Minnesota Histology and Research Laboratory in the Clinical and Translational Science Institute. Images were obtained on a Zeiss AxioCam IC at 200× magnification. Percentage medial thickness of small pulmonary arterioles was calculated as 100 times (outer diameter-inner diameter)/outer diameter using ImageJ.

Microtubule associated protein cosedimentation: Cosedimentation of microtubule-associated proteins (MAPs) in the RV was completed. In brief, frozen RV tissue was pulverized with a mortar and pestle cooled in liquid nitrogen and added to microtubule (MT) buffer (1% Triton X-100, 50 mM HEPES, 50 mM KCl, 1 mM MgCl2, 1 mM EGTA) and mammalian protease inhibitor (Sigma). Extracts were then incubated at 4 C for 1 hour and centrifuged at 100,000 g for 40 minutes at 25 C to remove sarcomeric proteins. 1 mM GTP and 1 mM DTT were added to the soluble fraction and incubated at 37° C. for 15 minutes. 20 μM taxol was subsequently added to the extract to stabilize microtubules. 300 μl of each fraction was then layered on cushion buffer (MT buffer plus 40% sucrose) and centrifuged at 100,000 g for 30 minutes at 25° C. The pellet fraction was resuspended in TMT isobaric label reagent (Thermo Scientific) and used for quantitative mass spectrometry analysis of MAPs.

Quantitative mass spectrometry: 18 μg of each pellet fraction was labeled with TMT10plex isobaric label reagent (Thermo Scientific) and mixed together in an equal ratio. Then, the multiplexed sample was cleaned with a 3 ml Sep-Pak C18 solid phase extraction cartridge (Waters Corporation, Milford, Mass.) and dried in vacuo. The cleaned sample was run on the Thermo Fusion. LC-MS data was acquired for each sample using an Easy-nLC 1000 HPLC (Thermo Scientific) in tandem with an Orbitrap Fusion (Thermo Scientific). Peptides were loaded directly onto a 75 cm×100-μm internal diameter fused silica PicoTip Emitter (New Objective, Woburn, Mass.) packed in-house with ReproSil-Pur C18-AQ. The column was mounted in a nanospray source directly in-line with an Orbitrap Fusion mass spectrometer (Thermo Scientific). Gas phase fractionation was employed via multiple LC-MS acquisition runs on the same sample to survey mass spectra on windows of 380-680, 680-980, 980-1280, 1280-1580, 380-1580 m/z with a resolution of 60,000 at 100 m/z with automatic gain control, 250-ms min injection time and lock mass at 445.1200 m/z (polysiloxane). The 12 most intense ions (2-7 charged state) from the full scan were selected for fragmentation by higher-energy collisional dissociation.

PAH patient cohort: PAH patients from the University of Minnesota Pulmonary Hypertension Program who had serum IL-6 levels measured were analyzed. PAH was defined as mean pulmonary arterial pressure (mPAP)>20 mmHg, pulmonary capillary wedge pressure <15 mmHg, and pulmonary vascular resistance (PVR)>3.0 Wood unit, not due other causes such as left-sided heart disease, chronic lung disease, or chronic thromboembolic disease as assessed by echocardiography, pulmonary function tests, computed tomography imaging, ventilation perfusion imaging, or invasive pulmonary angiogram. The serum N-terminal pro B-type natriuretic peptide (NT-proBNP) level, transthoracic echocardiogram, and right heart catheterization results closest to the date the IL-6 level were measured (average time difference was 2.1 months, median time difference 0.5 months). RV fractional area change was independently determined from transthoracic echocardiography. 73 PAH patients were assessed, 77% female, average age of 56.3 years at the time IL-6 levels were measured.

Statistical analysis: To compare means of two groups, unpaired t-test was used if the variance was similar but if there was unequal variance, the Mann-Whitney U-test was used. When comparing the means of three groups, one-way analysis of variance (ANOVA) with Tukey post-hoc analysis was completed when the variance was similar. If there was unequal variance between groups, Brown-Forsythe and Welch ANOVA with Dunnett post-hoc analysis were completed. Simple linear regression was used to evaluate whether there were any differences in the RV fractional area change (FAC) vs. mean pulmonary arterial pressure (mPAP) and RV FAC vs. pulmonary vascular resistance (PVR) relationships between patients with higher vs. lower IL-6 levels.

Statistical significance was defined asp-value <0.05. To analyze global changes in proteomic samples, principal component analysis (PCA) and hierarchical cluster analysis was performed with MetaboAnalyst. To evaluate the cellular function of the differentially expressed microtubule-associated proteins, network analysis was completed with Ingenuity Pathway Analysis. Statistical analysis and graphing were performed on GraphPad Prism version 8 except for the PCA and hierarchical cluster analysis described above. Values are presented as mean±standard error of mean (SEM). Graphs are depicted as bar graphs, Violin plots to display the distribution of the data, or box and whisker plots with the 10-90th percentile delineated and values that are out of those regions displayed as individual points.

Results

GP130 Antagonism Blunts STAT3 Activation and Normalizes Expression of the MT-JPH2 Pathway in the RV

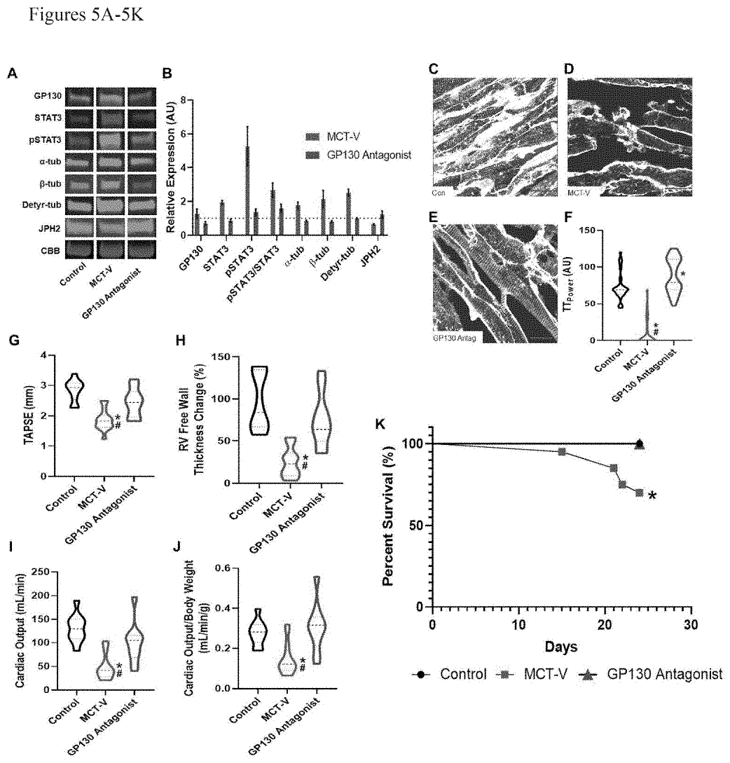

The effects of GP130 antagonism on STAT3 activity and the MT-JPH2 pathway were evaluated. GP130 antagonism decreased RV expression of GP130 (0.7±0.1 vs. 1.3±0.3 relative abundance), STAT3 (0.9±0.1 vs. 1.9±0.1 relative abundance), phosphorylated STAT3 (pSTAT3) (1.4±0.2 vs. 5.3±1.2 relative abundance), and the ratio of pSTAT3 to STAT3 (1.6±0.2 vs. 2.7±0.4 relative abundance) compared to MCTvehicle (MCT-V) rats (FIGS. 5A-5B). Furthermore, GP130 antagonism normalized the MT-JPH2 pathway. Expression of α-tubulin (0.8±0.1 vs. 1.8±0.2 relative expression), β-tubulin (0.8±0.1 vs. 2.1±0.5 relative expression), and detyrosinated α-tubulin, the form of tubulin found in stabilized microtubules (1.0±0.05 vs. 2.5±0.2 relative expression) were decreased and expression of JPH2 (1.2±0.2 vs. 0.6±0.04 relative expression) was increased compared to MCT-V RV (FIGS. 5A-5B).

GP130 Antagonism Restores t-Tubule Architecture, Improves RV Function, and Prevents Premature Mortality

GP130 antagonism completely normalized RV t-tubule architecture (TTpower 88.0±23.4 vs. 7.4±2.5 AU, p<0.0001) (FIGS. 5C-5F). These histological changes were associated with enhanced RV function as TAPSE (2.5±0.2 vs. 1.9±0.1 mm, p=0.004), percent change in RV free wall thickness (75.0±11.0 vs. 24.9±4.6%, p=0.005), cardiac output (100.6±14.9 vs. 49.1±7.3 mL/min, p=0.003), and cardiac output normalized to body weight (0.31±0.04 vs. 0.15±0.02 mL/min/g, p=0.0008) (FIGS. 5G-5J) were increased in GP130 rats compared to MCT-V. Hemodynamic studies showed that GP130 antagonism improved RV ejection fraction (88.0±2.6 vs. 63.3±3.3%, p<0.0001, N=8-14 animals per group) and trended in augmenting RV pulmonary artery (PA) coupling as assessed by the relationship between end-systolic elastance and effective arterial elastance (Ees/Ea) (0.91±0.44 vs. 0.44±0.29, p=0.06). Additionally, the augmented RV function in GP130 antagonist rats corresponded with improved survival rates (FIG. 5K) as there was a 30% mortality rate at the time of endpoint analysis (24 days after MCT or PBS injection) compared to a 0% mortality rate in the control and GP130 antagonist groups (p=0.006). Thus, GP130 antagonism restored the t-tubule architecture that resulted in improved RV function and protection from premature mortality.

Quantitative Mass Spectrometry Reveals a Link Between Microtubule Remodeling and Metabolic Dysregulation

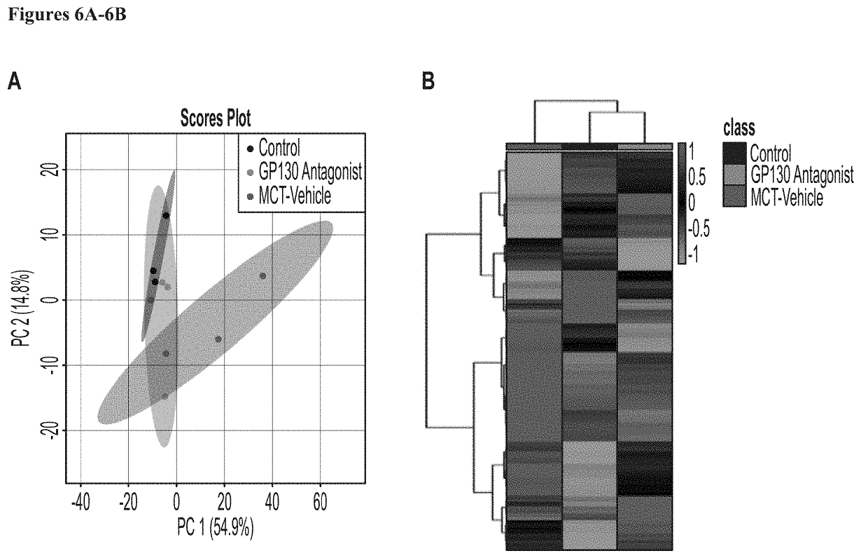

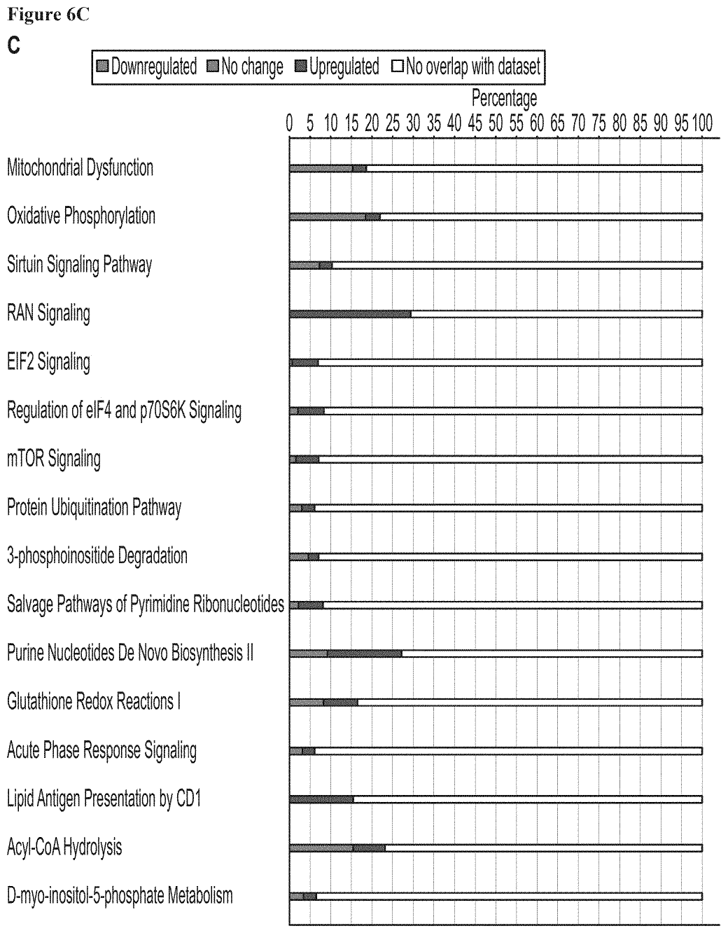

To further expand on the role of altered microtubule remodeling caused by GP130 blockage, the MAPs in control, MCT-V, and GP130 antagonist RVs were examined using quantitative mass spectrometry. 2842 proteins were identified with quantitative information in mass spectrometry analysis. GP130 antagonism restored the global expression signature of MAPs to a signature similar to control RV as depicted by PCA (FIG. 6A) and hierarchical cluster analysis (FIG. 6B). Pathway analysis of the significantly dysregulated MAPs showed an enrichment of proteins involved in mitochondrial dysfunction and oxidative phosphorylation (FIG. 6C), providing a potential link between microtubule remodeling and metabolic derangements.

GP130 Antagonism Decreases RV Hypertrophy and Fibrosis

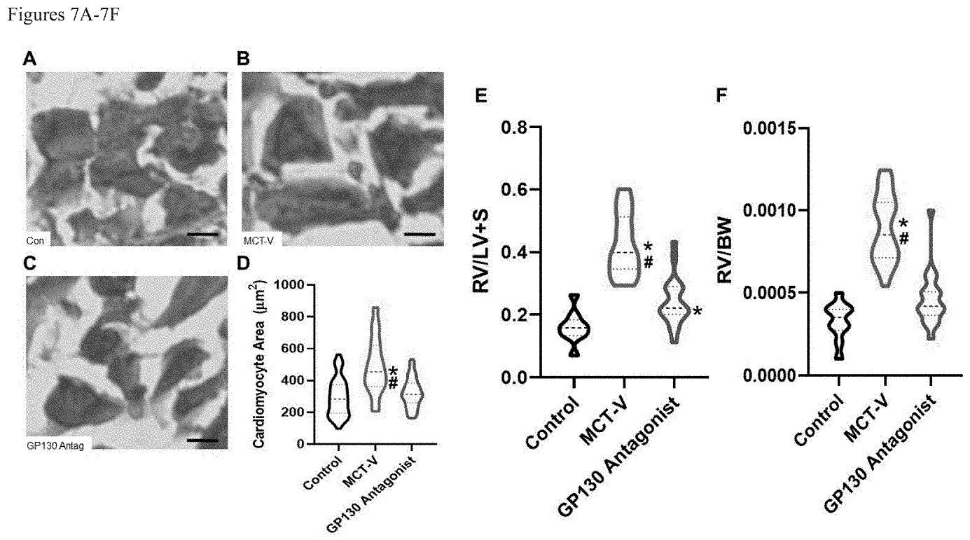

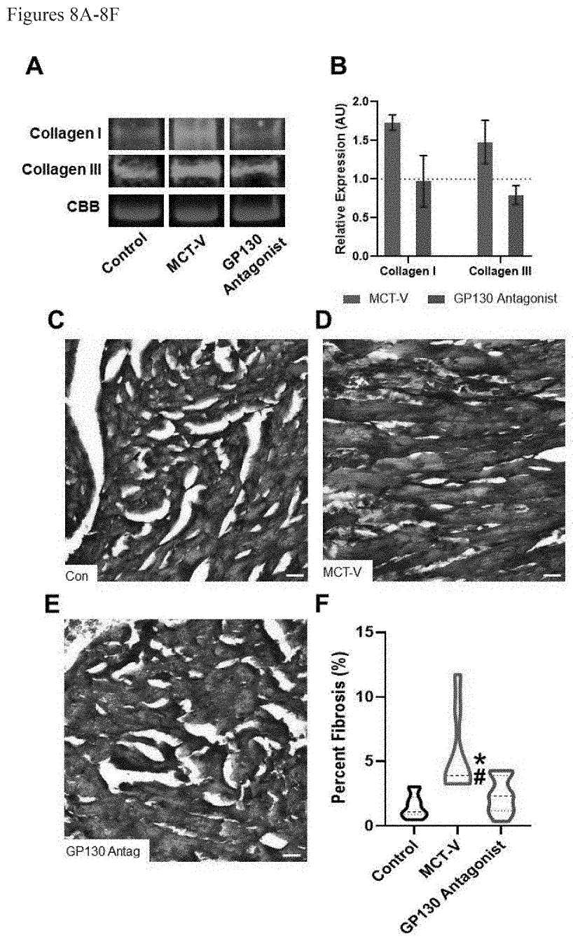

Next, RV size and histology were evaluated. GP130 antagonism decreased RV cardiomyocyte area compared to MCT-V (323.0±10.5 vs. 492.5±24.0 μm2, p<0.0001) (FIG. 7A-7D). Furthermore, the Fulton index (0.24±0.02 vs. 0.42±0.02, p<0.0001) (FIG. 7E) and RV size normalized to body weight (0.00046±0.00003 vs. 0.00089±0.00004, p<0.0001) (FIG. 7F) were reduced in GP130 rats compared to MCT-V. To evaluate if fibrosis also played a role in the differences, levels of collagen I and III were examined. Rats treated with the GP130 antagonist had decreased expression of collagen I (1.0±0.3 vs. 1.7±0.1 relative abundance) and collagen III (0.8±0.1 vs. 1.5±0.3 relative abundance) in the RV compared to MCT-V rats (FIG. 8A-8B). These expression changes correlated with histological changes as GP130 antagonism decreased RV fibrosis compared to MCT-V on Masson's trichrome staining (2.3±0.4 vs. 5.5±1.1%, p=0.003) (FIG. 8C-8F). Collectively, these results demonstrate that GP130 antagonism decreases RV hypertrophy and fibrosis.

Improvements in RV Structure and Function Occur Independent of Changes in the Pulmonary Vasculature

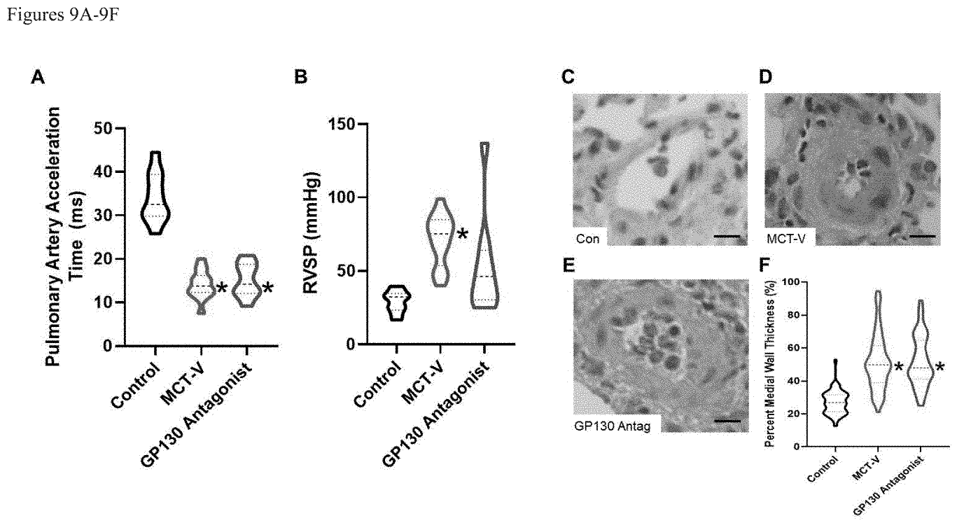

It was investigated whether changes in RV afterload led to the improvements in RV function in the GP130 antagonist rats. No difference in pulmonary artery acceleration time was observed between GP130 and MCT-V animals (15.0±1.3 vs. 14.1±0.9 ms, p=0.87) (FIG. 9A). Additionally, when invasive hemodynamics were evaluated, there were no significant differences in right ventricular systolic pressure (RVSP) between GP130 and MCT-V rats (53.0±11.6 vs. 71.0±4.3 mmHg; p=0.13) (FIG. 9 B). Furthermore, there were no differences in percent medial wall thickness of the small pulmonary arterioles between MCT-V and GP130 antagonist rats (51.6±1.7 vs. 52.0±1.6%, p=0.996) (FIG. 9C-9F). These findings demonstrate that GP130 antagonism improves RV structure and function independent of changes to the pulmonary vasculature.

Higher IL-6 Levels in PAH Patients is Associated with Worse RV Function Independent of Changes in the Pulmonary Vasculature

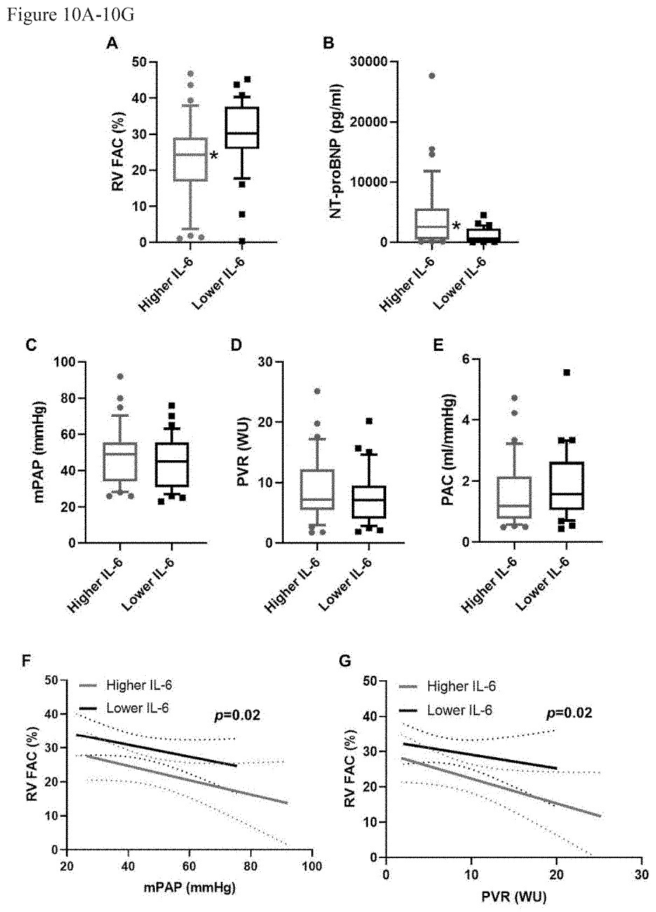

It was next assessed whether serum IL-6 levels were related to RV function in PAH patients. The patient cohort was stratified by the median IL-6 level, and patients with higher IL-6 levels had indicators of worse RV function as assessed by lower RV fractional area change (FAC) on echocardiography (23±2 vs. 30±2%, p=0.005) and higher NT-proBNP levels (4192±947 vs. 1197±192 pg/ml, p=0.001) (FIG. 10A-10B). There were no changes in pulmonary vasculature parameters such as mean pulmonary arterial pressure (mPAP) (48±3 vs. 45±3 mmHg, p=0.35), pulmonary vascular resistance (PVR) (9.2±1.0 vs. 7.5±0.8 WU, p=0.18), or pulmonary arterial compliance (PAC) (1.6±0.2 vs. 1.9±0.2 ml/mmHg, p=0.32) (FIG. 6C-E). Moreover, when the relationship between RV FAC and mPAP was examined, patients with higher IL-6 levels had lower RV FAC at each mPAP compared to patients with lower IL-6 levels (p=0.02) (FIG. 10F). Similarly, when the relationship between RV FAC and PVR was evaluated, patients with higher IL-6 levels had lower RV FAC at each PVR compared to patients with lower IL-6 (p=0.02) (FIG. 6G). These data suggest that excess GP130 signaling in human PAH is associated with worse RV function.

Discussion

RV dysfunction is the leading cause of death in PAH patients, and finding therapies that improve RV function is critical to combat this deadly disease. In this study, it was demonstrated that GP130 signaling via STAT3 regulated the MT-JPH2 pathway in the RV in PAH. Additionally, chemical inhibition of GP130 normalized the MT-JPH2 pathway in the RV of MCT rats, reversed pathological t-tubule remodeling, augmented RV function, decreased RV hypertrophy and fibrosis, and enhanced survival independent of changes to the pulmonary vasculature or improvement in pulmonary hypertension. Quantitative proteomics analysis of RV MAPs showed near normalization of the proteomic signature with GP130 antagonism and pathway analysis of differentially expressed MAPs revealed enrichment of proteins in mitochondrial and metabolic pathways, suggesting a link between microtubule remodeling and mitochondrial function. Finally, in 73 PAH patients, it was shown that elevated serum levels of IL-6 were associated with more severe RVD despite similar hemodynamic severity of PAH, validating previous findings with a larger cohort in the present study.

The importance of the MT-JPH2 pathway and t-tubule remodeling in the pathogenesis of heart failure are well documented, and thus our results are congruent with previous studies. A previous study showed MT-mediated JPH2 disorganization leads to t-tubule disruption and left ventricular (LV) failure in pressure overload. Furthermore, adeno-associated virus type 9 mediated overexpression of JPH2 prevents loss of tubules and improves LV function in pressure-overloaded mice. Furthermore, in addition to modulating JPH2 expression and localization, microtubules themselves can modulate cardiac contraction. Previous studies show greater microtubule density and excess tubulin detyrosination increases diastolic stiffness and reduces systolic function. Thus, it is likely that the MT-JPH2 pathway plays a major role in the improvement of RV function with GP130 antagonism.

The direct link between GP130 signaling and microtubule remodeling is likely pleiotropic as previous chromatin immunoprecipitation experiments demonstrate STAT3 regulates expression of multiple isoforms of α- and β-tubulin, and MAP4, a microtubule stabilizing protein. Furthermore, there is evidence that STAT3 binds to and inhibits the activity of stathmin, a protein that binds to and depolymerizes microtubules. However, stathmin is not highly expressed in cardiac muscle. Moreover, there is evidence that STAT3 can directly bind microtubules. However, the proteomics data did not reveal differences in STAT3 levels in the MAP fraction between the three groups analyzed. Thus, it is most likely that STAT3 transcriptional modulation of tubulin isoforms and the microtubule stabilizing protein, MAP4 is promoting microtubule stabilization in cardiomyocytes.

The finding that GP130 antagonism modulates RV fibrosis is supported by multiple previous publications. First, genetic deletion of IL-6 reduces interstitial fibrosis in diabetic cardiomyopathy. Moreover, inhibition of STAT3 activation with parthenolide reduces LV fibrosis via modulation of fibroblast activation. Finally, deletion of βIV-spectrin in fibroblasts promotes STAT3 signaling which ultimately causes cardiac dysfunction. Importantly, small molecule inhibition of STAT3 reverses cardiac dysfunction caused by βIV spectrin deletion. Clearly, there are multiple lines of evidence linking STAT3 signaling to pathological cardiac fibrosis, which may explain our finding that GP130 antagonism blunts RV fibrosis to augment RV function.

Certainly, the proteomics data that provide a link between microtubule remodeling and mitochondrial function require further exploration. However, previous studies show the microtubular network and MAPs can affect mitochondrial morphology and motility. Additionally, many MAPs are metabolic enzymes. Another possible link between microtubules and mitochondria is suggested by data from fission yeast showing that the physical association of mitochondria with microtubules prevents mitochondrial fission via inhibition of binding of Dnm134, the yeast analogue of dynamin-related protein (DRP). Although excess mitochondrial fission promotes RV dysfunction in the monocrotaline rat model of PAH35, complete inhibition of mitochondrial fission is also deleterious to cardiac function as cardiac-specific genetic deletion of DRP-1 induces cardiomyopathy and premature mortality.

The clinical trial investigating the effects of tocilizumb, a monoclonal antibody that antagonizes the IL-6 receptor, in the treatment of PAH (TRANSFORM-UK, NCT02676947) was recently completed. The primary outcomes of the trial were safety and PVR and NT-proBNP was a secondary outcome. The results of TRANSFORM-UK have not yet been published, but PVR was not different between the tocilizumab and control groups. The present results recapitulate this finding as we did not see significant changes in pulmonary vascular disease with GP130 antagonism (FIGS. 9A-9F). Moreover, no differences in pulmonary vascular disease severity between PAH patients with high compared to low serum IL-6 levels (FIG. 10C-10E) was observed. Perhaps the link between IL-6 and higher mortality in PAH may be due to worse RV function and not pulmonary vascular disease severity. Evaluation of RV function in patients with elevated IL-6 treated with tocilizumab may support this hypothesis, but unfortunately RV function was not specifically analyzed in TRANSFORM-UK.

Conclusions

Antagonism of GP130 signaling enhances RV function independent of changes in the pulmonary vasculature. Modulation of pathological microtubule remodeling plays a key role in the beneficial effects of GP130 antagonism.

### Example 4

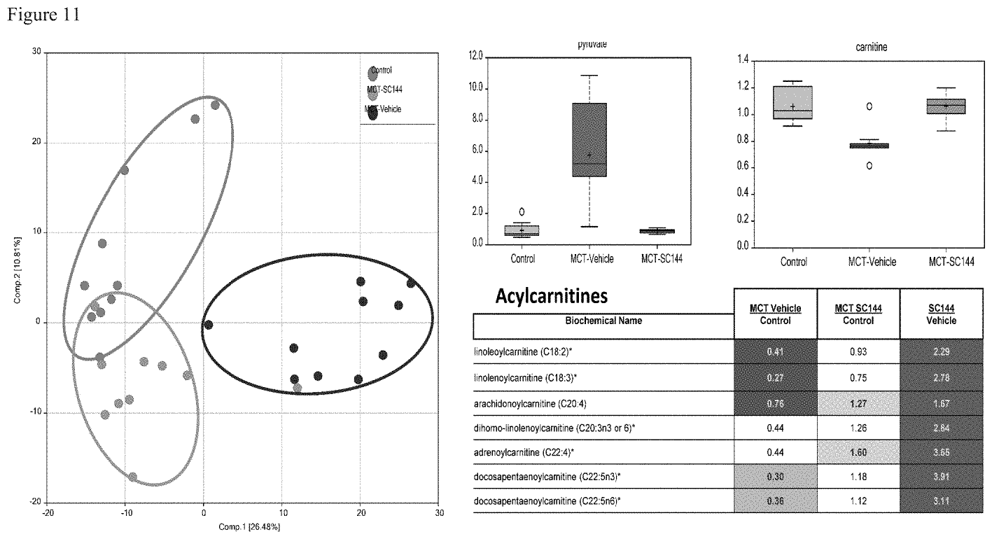

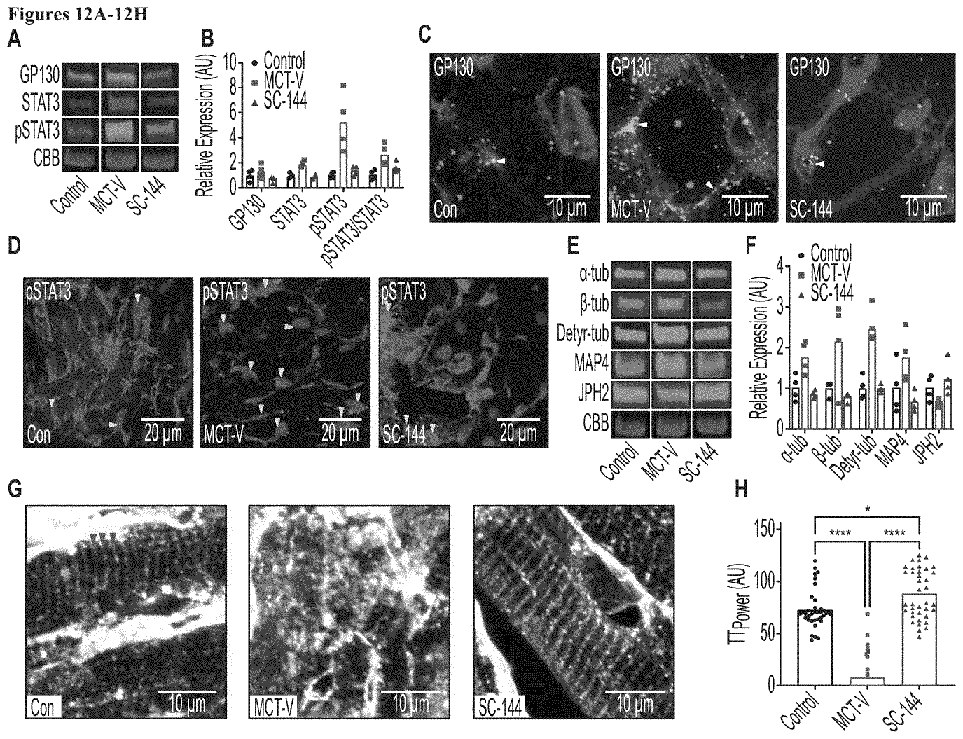

Metabolomics data showing that SC-144 is able to restore the metabolic signature of the right ventricle in rats with PAH (FIG. 11). Global metabolomics profiling from right ventricular extracts revealed an improvement in the metabolic profile of the rats treated with SC-144. On the left, the principal component analysis revealed a shift in the metabolic signature towards controls in the rats treated with SC144. On the right, there is evidence of a shift away from anaerobic glycolysis with a normalization of pyruvate levels, suggesting improved mitochondrial function. Moreover, there is evidence of enhanced acylcarnitine metabolism as total carnitine levels are normalized with SC144. Finally, individual acylcarnitine levels are reduced in the PAH model, but SC144 treatment helps restore multiple acylcarnitine levels in the RV. These data provide evidence that SC-144 is able to combat pathological metabolic remodeling in the RV.

### Example 5

Abstract

Background: Right ventricular dysfunction (RVD) is the leading cause of death in pulmonary arterial hypertension (PAH), but no RV-specific therapy exists. We showed microtubule-mediated junctophilin-2 dysregulation (MT-JPH2 pathway) causes t-tubule disruption and RVD in rodent PAH, but the druggable regulators of this critical pathway are unknown. Glycoprotein-130 (GP130) activation induces cardiomyocyte microtubule remodeling in vitro, however the effects of GP130 signaling on the MT-JPH2 pathway and RVD resulting from PAH are undefined.

Methods: Immunoblots quantified protein abundance, quantitative proteomics defined RV microtubule-associated proteins (MAPs), metabolomics evaluated the RV metabolic signature, and transmission electron microscopy (TEM) assessed RV cardiomyocyte mitochondrial morphology in control, monocrotaline (MCT), and MCT-SC-144 (GP130 antagonist) rats. Echocardiography and pressure-volume loops defined the effects of SC-144 on RV-pulmonary artery coupling in MCT rats. In 73 PAH patients, the relationship between interleukin-6, a GP130 ligand, and RVD was evaluated.

Results: SC-144 decreased GP130 activation, which normalized MT-JPH2 protein expression and t-tubule structure in the MCT RV. Proteomics analysis revealed SC-144 restored RV MAP regulation. Ingenuity pathway analysis of dysregulated MAPs identified a link between microtubules and mitochondrial dysfunction. Specifically, SC-144 prevented dysregulation of electron transport chain, Kreb's cycle, and the fatty acid oxidation pathway proteins. Metabolomics profiling suggested SC-144 reduced glycolytic dependence, glutaminolysis induction, and enhanced fatty acid metabolism. TEM and immunoblots indicated increased mitochondrial fission in the MCT RV, which SC-144 mitigated. GP130 antagonism reduced RV hypertrophy and fibrosis and augmented RV-pulmonary artery coupling without alter PAH severity. In PAH patients, higher interleukin-6 levels were associated with more severe RVD.

Conclusions: SC144 combatted MT-JPH2 dysregulation, metabolic derangements, and RVD in MCT rats. Thus, GP130 antagonism could be a RV-targeted therapeutic in PAH.

Introduction

Pulmonary arterial hypertension (PAH) is a progressive vasculopathy that increases pulmonary arterial pressures and reduces pulmonary arterial compliance. The pathological alterations in the pulmonary circuit ultimately manifest as right ventricular dysfunction (RVD). Although RVD is the strongest predictor of mortality in PAH, the molecular mediators of RVD are understudied. This knowledge gap may explain the absence of pharmaceuticals that directly combat RVD pathophysiology. Unfortunately, medications used for left ventricular (LV) failure have not yielded similar success in RVD. Thus, there is an urgent need to develop RV-directed therapies to enhance quality of life and improve survival in PAH.

We previously demonstrated the importance of microtubules in preclinical RVD via their regulation of junctophilin-2 (the MT-JPH2 pathway). In monocrotaline (MCT) PAH, there is a chamber-specific microtubule stabilization in RV cardiomyocytes that lowers JPH2 levels and leads to abnormalities in the structure of t-tubules that impair RV contractility. Importantly, colchicine-mediated microtubule depolymerization increases JPH2 levels, combats pathological t-tubule remodeling, and augments RV function in MCT rats, a validated model of RV failure. Xie et al. showed the degree of JPH2 reduction and t-tubule derangements correlates with the severity of RV failure in MCT rats. Certainly, the MT-JPH2 pathway is critical for RV cardiomyocyte function via its impact on modulation of t-tubule structure, calcium handling, and cardiomyocyte contractility. Unfortunately, our colchicine results may not readily translate to human RVD because the equivalent human dose may have toxicities. In order to capitalize on the therapeutic potential of restoring the MT-JPH2 pathway, we chose to focus on upstream regulators in hopes of identifying a druggable molecular target for RVD.

Glycoprotein-130 (GP130) is the master membrane receptor of the interleukin-6 (IL-6) superfamily of cytokines. GP130 downstream signaling molecules include the Janus kinase/signal transducer and activator of transcription (JAK/STAT), phosphatidylinositol 3-kinase (PI3K), and mitogen activated protein kinase (MAPK) pathways. However, STAT3 is currently believed to be the predominant intracellular effector protein. GP130 may have direct relevance to the MT-JPH2 pathway as GP130 stimulation stabilizes microtubules in neonatal cardiomyocytes. Importantly, STAT3 phosphorylation is essential for GP130-mediated microtubule remodeling. At present, the role of GP130 signaling in RVD due to PAH is undefined. However, clinical studies suggest the biological plausibility that GP130 might modulate RV function as IL-6 levels are independently associated with RVD in PAH patients. Moreover, multiple PAH cohort studies show that elevated IL-6 levels predict worse survival rates despite minimal differences in pulmonary vascular disease severity.

Remodeling of cardiomyocyte microtubules alters t-tubule integrity and contractility, but the detrimental effects of microtubule perturbations on other areas of cardiomyocyte cell biology are unexplored. Chemical modulation of microtubules affects the balance of mitochondrial fission and fusion in budding yeast, which ultimately results in mitochondrial dysfunction. This finding may be applicable to the RV because excess mitochondrial fission promotes RVD in rodent PAH. A recent transcriptomic study of the RV in MCT rats and human PAH identifies mitochondrial metabolic dysfunction and inflammation as the two most dysregulated pathways in both species, but the molecules that promote crosstalk between these two pathological entities are unknown. We speculate GP130-induced microtubule dysregulation mediates the intersection of inflammation and the acquired imbalance of mitochondrial fission/fusion and subsequent metabolic dysfunction of the RV in PAH.

In this study, we hypothesized increased GP130 signaling induces pathological microtubule remodeling, which causes RVD by induction of JPH2 downregulation and mitochondrial metabolic dysfunction. We used a small molecule GP130 antagonist, starting two weeks after MCT injection, to reverse microtubule-mediated JPH2 downregulation and t-tubule disruption in MCT rats. In addition, we employed quantitative proteomics to define the effects of GP130 antagonism on the microtubule-associated protein (MAP) fraction of the RV. We also used transmission electron microscopy (TEM) and global metabolomics profiling to determine how GP130-mediated microtubule remodeling altered mitochondrial structure and metabolic function. We subsequently assessed how inhibition of GP130 signaling affected RV-pulmonary artery coupling using both echocardiography and high-fidelity cardiac catheterization generated pressure-volume loops. Finally, we examined the relationship between serum IL-6 levels and RV function in human PAH. Our findings establish a role for GP130 signaling in RVD and show that this inflammatory pathway dysregulates mitochondrial form and function which contributes to RVD. Finally, we provide promising preclinical data supporting GP130 as a potential RV-specific target in treating RVD in PAH.

Methods

Briefly, male Sprague Dawley rats received a single subcutaneous injection of MCT or phosphate buffered saline. Two weeks after MCT injection (60 mg/kg), rats received either daily intraperitoneal injections of the GP130 antagonist, SC-144 (10 mg/kg), or a dimethyl sulfoxide and propylene glycol vehicle for 10 days. Animal studies were approved by the University of Minnesota Institutional Animal Care and Use Committee and followed best practices for preclinical studies. Immunoblots quantified levels of cardiac protein abundance as previously described. Antibodies used are described in Table 2.

TABLE 2
Antibodies used in study.

| | | | | |
|---|---|---|---|---|
| | | | Catalogue | |
| Antigen | Company | Species | Number | Dilution |

Western Blot

| | | | | |
|---|---|---|---|---|
| GP130 | Cell Signaling | Rabbit | 3732 | 1:250 |
| | Technology | | | |
| Stat3 | Cell Signaling | Mouse | 9139 | 1:250 |
| | Technology | | | |
| P-Stat3 (Y705) | Abcam | Rabbit | ab76315 | 1:250 |
| α-tubulin | Millipore | Mouse | 05-829 | 1:250 |
| β-tubulin | Sigma-Aldrich | Mouse | T4026 | 1:250 |
| Detyrosinated α- | Abcam | Rabbit | ab48389 | 1:250 |
| tubulin | | | | |
| MAP4 | Millipore | Rabbit | ab6020 | 1:250 |
| JPH2 | Invitrogen | Rabbit | 405300 | 1:250 |
| MFN1 | Abcam | Mouse | ab57602 | 1:250 |
| MFN2 | Abcam | Mouse | ab56889 | 1:250 |
| OPA1 | BD Biosciences | Mouse | 612606 | 1:250 |
| FIS1 | Sigma-Aldrich | Rabbit | HPA017430 | 1:250 |
| DRP1 | BD Biosciences | Mouse | 611112 | 1:250 |
| Collagen I | Abcam | Rabbit | ab34710 | 1:250 |
| Collagen III | Abcam | Rabbit | ab6310 | 1:100 |
| Anti-mouse | Li-COR | Goat | 926-32210 | 1:5000 |
| secondary | | | 926-68070 | |
| Anti-rabbit | Li-COR | Goat | 926-32211 | 1:5000 |
| secondary | | | 926-68071 | |

Immunofluorescence

| | | | | |
|---|---|---|---|---|
| GP130 | Cell Signaling | Rabbit | 3732 | 1:50 |
| | Technology | | | |
| P-Stat3 (Y705) | Abcam | Rabbit | ab76315 | 1:50 |
| Anti-rabbit | Invitrogen | Goat | A32732 | 1:500 |
| secondary, Alexa | | | | |
| Fluor plus 555 | | | | |
| Wheat germ | Invitrogen | | W21404 | 1:50 |
| agglutinin, Alex | | | | |
| Fluor 633 | | | | |
| conjugate | | | | |

Co-sedimentation of MAPs in RV extracts was completed. 875 μg of MAP extract was layered on cushion buffer and centrifuged at 100,000 g for 30 minutes at 25° C. The pellet fraction (microtubules and MAPs) was resuspended in TMT isobaric label reagent (Thermo Scientific) and used for quantitative mass spectrometry analysis. Methods for quantitative mass spectrometry are described below.

Metabolomic profiling of frozen RV free wall specimens was performed by Metabolon Inc. (Durham, N.C.). Ultrahigh performance liquid chromatography/electrospray ionization tandem mass spectrometry was conducted on the Discovery HD4™ Global Metabolomics platform and results were provided as semi-quantitative metabolite levels.

Echocardiography, pressure-volume loops, and pulmonary vascular histology assessed the effects of SC-144 on RV function and pulmonary vascular disease severity. Finally, we examined the relationship between the GP130 agonist, IL-6 and RVD in 73 PAH patients (Table 3).

TABLE 3
Characteristics of PAH cohort.

| | Total Cohort | Higher IL-6 | Lower IL-6 |
|---|---|---|---|
| Characteristics | (n = 73) | Group (n = 36) | Group (n = 37) |
| Age (years) | 56.3 ± 14.8 | 55.9 ± 14.9 | 56.7 ± 14.9 |
| Female sex | 56 (77%) | 27 (75%) | 29 (78%) |
| Etiology | | | |
| Idiopathic PAH | 23 (31.5%) | 7 (19.4%) | 16 (43%) |
| Heritable PAH | 1 (1.4%) | 1 (2.8%) | 0 (0%) |
| Drug-induced PAH | 3 (4%) | 2 (5.6%) | 1 (3%) |
| CTD-associated PAH | 33 (45.2%) | 18 (50%) | 15 (41%) |
| HIV-associated PAH | 1 (1.4%) | 1 (2.8%) | 0 (0%) |
| Portopulmonary PAH | 7 (9.6%) | 5 (13.8%) | 2 (5%) |
| Congenital heart disease | 4 (5.5%) | 2 (5.6%) | 2 (5%) |
| PVOD | 1 (1.4%) | 0 (0%) | 1 (3%) |
| Median IL-6 level [range] (pg/mL) | 5.28 [0.95-119] | 12.86 [5.29-119] | 2.56 [0.95-5.28] |

Data presented as mean±standard deviation except for the IL-6 level, which is reported as the median with the range since it is not normally distributed.

Supplemental Methods:

Animal models: Male Sprague Dawley rats (200-250 g; 7-8 weeks old) (Charles River Laboratories, Wilmington, Mass.) received a single subcutaneous injection of monocrotaline (MCT) (60 mg/kg) (Sigma-Aldrich, St. Louis, Mo.) or phosphate-buffered saline (control). Two weeks after MCT injection, rats received daily intraperitoneal injections of the GP130 antagonist, SC-144 (10 mg/kg) (ApexBio, Houston, Tex.), or vehicle for 10 days and then endpoint analysis was completed. SC-144 was dissolved in dimethyl sulfoxide (DMSO) to a concentration of 200 mg/mL, diluted to 20 mg/mL in propylene glycol, and further added to 0.9% NaCl and 40% propylene glycol to achieve a final concentration of 10 mg/kg. Rats were randomized to treatment groups. We estimated a sample size of 15-20 rats per group to achieve statistical significance. Animal studies were approved by the University of Minnesota Institutional Animal Care and Use Committee.

Western blot analysis: Immunoblots with 25 μg of RV protein extracts were performed using the Odyssey Infrared Imaging system (Lincoln, Nebr.). Post transfer SDS-PAGE gels were stained with Coomassie brilliant blue (CBB) and imaged at the 700-nm wavelength on the Odyssey Imaging System with the band corresponding to the myosin heavy chain used as the loading control. Antibodies used in this study are listed in Table 2.

RV Tissue Microtubule Cosedimentation Assay: Cosedimentation of MAPs in RV extracts was completed. In brief, frozen RV tissue was pulverized with a mortar and pestle cooled in liquid nitrogen and added to microtubule (MT) buffer (1% Triton X-100, 50 mM HEPES, 50 mM KCl, 1 mM MgCl₂, 1 mM EGTA) and mammalian protease inhibitor (Sigma). Extracts were solubilized at 4° C. for 1 hour and centrifuged at 100,000 g for 40 minutes at 4° C. Guanosine triphosphate and 1,4-dithiotheritol (1 mM each) were added to the soluble fraction and incubated at 37° C. for 15 minutes to induce microtubule polymerization. 20 μM paclitaxel (Cytoskeleton, Denver, Colo.) was subsequently added to stabilize microtubules. 875 μg of extract was layered on cushion buffer (MT buffer plus 40% sucrose) and centrifuged at 100,000 g for 30 minutes at 25° C. The pellet fraction (microtubules and MAPs) was resuspended in TMT isobaric label reagent (Thermo Scientific) and used for quantitative mass spectrometry analysis

Quantitative mass spectrometry: 18 μg of each MAP pellet was labeled with TMT10plex isobaric label reagent (Thermo Scientific) and mixed together in an equal ratio. The multiplexed sample was cleaned with a 3 ml Sep-Pak C18 solid phase extraction cartridge (Waters Corporation, Milford, Mass.) and dried in vacuo. The sample was run on the Thermo Fusion. LC-MS data was acquired for each sample using an Easy-nLC 1000 HPLC (Thermo Scientific) in tandem with an Orbitrap Fusion (Thermo Scientific).

Electron microscopy: RV free wall samples approximately 1-2 mm³ were fixed in 3% paraformaldehyde, 1.5% glutaraldehyde, and 2.5% sucrose in 0.1 M sodium cacodylate buffer with 5 mM calcium chloride and 5 mM magnesium chloride (pH 7.4). The samples were subsequently rinsed in sodium cacodylate buffer three times for 10 minutes and stored in 1% osmium tetroxide and 0.1 M sodium cacodylate buffer (pH 7.4) overnight at 4° C. The RV specimens were rinsed in ultrapure water (NANOpure Infinity, Barnstead/Thermo Fisher Scientific, Waltham, Mass.), en bloc stained with 1% aqueous uranyl acetate for two hours, and rinsed in ultrapure water. The samples were then dehydrated in an ethanol series and embedded in Embed 812 resin (Electron Microscopy Sciences, Hatfield, Pa.). Ultrathin sections 80-100 nm thick were cut on a Leica Ultracut UCT microtome using a diamond knife and collected on formvar/carbon-coated copper slot (2 mm×1 mm) grids (Electron Microscopy Sciences). They were then stained with 3% aqueous uranyl acetate for 20 minutes, rinsed in ultrapure water, stained with Sato's triple-lead stain, and rinsed in ultrapure water. Sections were examined with a JEOL JEM1400-Plus transmission electron microscope operating at 60 and 120 kV. Images were recorded with a Maxim DL digital capture system. FIJI was used to measure mitochondrial area and length (across the longest axis) and width (the axis perpendicular to the longest axis). Variability in mitochondrial area was calculated by averaging the difference in mitochondrial area between each mitochondria and the smallest mitochondria measured for each respective animal.

Cell culture and mitochondrial respiration measurements: H9c2 (ATCC CRL-1446) rat cardiomyocytes were maintained in Dulbecco's Modified Eagle's Medium (DMEM) (Thermo Scientific) supplemented with 10% fetal bovine serum (FBS) (Foundation, Gemini Bio-Products, West Sacramento, Calif.), 100 U/ml penicillin, and 100 μg/ml streptomycin (Invitrogen, Carlsbad, Calif.) at 37° C. with 5% CO₂. Cells were passaged with Accutase (BioLegend, San Diego, Calif.). Differentiation was induced by changing to differentiation medium which had 2% horse serum (Equitech-Bio, Kerrville, Tex.), in place of FBS. Ten thousand cells were plated per Agilent Seahorse XFp dish pretreated with 0.1% gelatin (Sigma-Aldrich, St. Louis, Mo.). Five days after initial plating, cells were treated with differentiation media supplemented with 20 μM paclitaxel in DMSO (Sigma-Aldrich) overnight. The following day and one hour before the assay, cell media was changed to Seahorse DMEM assay media and supplemented with 5 mM glucose, 4 mM glutamine, and 1 mM pyruvate (Agilent, Santa Clara, Calif.). Oxygen consumption rate (OCR) was measured with an Agilent Seahorse XFp Extracellular Flux analyzer using the Agilent XFp Cell Mito Stress Test Kit with oligomycin A (final concentration after injection 1.5 μM), carbonyl cyanide 4-(trifluoromethoxy)phenylhydrazone (FCCP at 2 μM), and 0.5 mM rotenone/actimycin. After the assay, cells were lysed in 50 μl of 1× Laemmli sample buffer supplemented with HALT protease and phosphatase inhibitor cocktail (Thermo Fisher). Cell lysates were electrophoresed on SDS-PAGE gels and stained with CBB per above to estimate total protein per sample. Raw Seahorse measurements were normalized to total protein and analyzed using Agilent Wave software.

Cardiac histology: For the t-tubule analyses, 10-μm RV free wall cryosections were fixed in 4% paraformaldehyde, washed twice with PBS for 5 minutes, and incubated with Alexa Fluor-633 conjugated wheat germ agglutinin (Sigma-Aldrich, St. Louis, Mo.) for 30 minutes. Sections were mounted in Anti-Fade Reagent (Molecular Probes, Eugene, Oreg.). Images were obtained using an Olympus FV1000 BX2 upright confocal microscope (Tokyo, Japan) at the University of Minnesota Imaging Center. Z-stack images were converted into a Z-project using FIJI (National Institutes of Health, Bethesda, Md.), and t-tubule structure and regularity were quantified with the TTorg plugin on FIJI.

For the cardiomyocyte and fibrosis analyses, 10-μm RV cryosections were stained with hematoxylin and eosin (H&E) (Vector Laboratories, Burlingame, Calif.) or trichrome stain (Abeam, Cambridge, Mass.), respectively, per manufacturer's protocol. Images were obtained on a Zeiss AxioCam IC (Oberkochen, Germany). Cardiomyocyte area and percent fibrosis (determined by measuring the area of tissue stained blue divided by total tissue area) were assessed with FIJI.

Lung histology: Lung sections were fixed in 10% formalin, embedded in paraffin, sectioned at 4-μm, and stained with H&E by the University of Minnesota Histology and Research Laboratory in the Clinical and Translational Science Institute. Images were obtained on a Zeiss AxioCam IC. Percentage medial thickness of small pulmonary arterioles was calculated as 100 times (outer diameter-inner diameter)/outer diameter using FIJI.

Rodent echocardiography: Echocardiography was performed using a Vevo2100 ultrasound system with a 37.5-MHz transducer (VisualSonics). M-mode and 2-D modalities were used to measure tricuspid annular plane systolic excursion (TAPSE) and RV free wall (RVFW) thickness during end diastole and end systole. Pulmonary artery (PA) diameter was measured at the level of the pulmonary outflow tract during mid-systole. Pulsed-wave Doppler measured PA flow velocity time integral. Cardiac output was calculated.

Invasive closed-chest RV pressure-volume (PV) loops: Rats were anesthetized initially with 5% isoflurane induction and then maintained on 2-3% isoflurane. During the catheterization, rats were ventilated with the SomnoSuite small animal anesthesia system (Kent Scientific, Torrington, Conn.). A high-fidelity catheter (Scisense 1.9F pressure-volume, Transonic Systems, Ithaca, N.Y.) was advanced into the RV via the right internal jugular vein and right atria. RV pressure and volume were continuously recorded using Transonic AV500 Pressure-Volume Measurement System and subsequently analyzed on LabScribe version 4 (iWorx Systems, Dover, N.H.).

PAH patient cohort: PAH patients, defined as mean pulmonary arterial pressure (mPAP)≥20 mmHg, pulmonary capillary wedge pressure ≤15 mmHg, and pulmonary vascular resistance (PVR)≥3.0 Wood units, not due other causes such as left-sided heart disease, chronic lung disease, or chronic thromboembolic disease as assessed by echocardiography, pulmonary function tests, computed tomography imaging, ventilation perfusion imaging, or invasive pulmonary angiogram, from the University of Minnesota Pulmonary Hypertension Program were examined. We evaluated serum N-terminal pro B-type natriuretic peptide (NT-proBNP) level, RV fractional area change (RVFAC) from transthoracic echocardiogram, and right heart catheterization results closest to the date the IL-6 level was measured (average time difference was 2.1 months, median time difference 0.5 months). RVFAC was independently determined from offline transthoracic echocardiogram images by SZP and KWP. We assessed 73 PAH patients, 77% female, average age of 56.3 years at the time IL-6 levels were measured.

Statistical analysis: To compare the means of two groups, unpaired t-test was used if the variance was similar as determined by F-test, but if there was unequal variance, the Mann-Whitney U-test was completed. When comparing the means of three groups, one-way analysis of variance (ANOVA) with Tukey post-hoc analysis was used when the variance was similar. If there was unequal variance between groups, Brown-Forsythe and Welch ANOVA with Dunnett post-hoc analysis were completed. Linear regression evaluated whether there were differences in the slopes and y-intercepts of the best fit lines of RVFAC vs. mPAP and RVFAC vs. PVR relationships between patients with higher vs. lower IL-6 levels. Statistical significance was defined asp-value <0.05. To analyze global changes in proteomic samples, principal component analysis and hierarchical cluster analysis were performed with MetaboAnalyst⁵⁷ software. To evaluate the cellular function of differentially expressed MAPs (defined using Benjamini Hochberg procedure), network analysis was completed with Ingenuity Pathway Analysis (Qiagen, Hilden, Germany). Statistical analysis and graphing were performed on GraphPad Prism version 9 except for the principal component analysis plots and hierarchical cluster analyses described above. Values are presented as mean±standard error of mean (SEM) or are specified otherwise. Graphs are depicted as bar graphs with the mean and all individual values.

Results

SC-144 Mitigated GP130 Activation in the MCT RV

First, we investigated how MCT-PAH affected GP130 pathway activation in the RV. MCT-Vehicle (MCT-V) rats had elevated expression of GP130, STAT3, phosphorylated STAT3 (pSTAT3), and the ratio of pSTAT3 to STAT3 as compared to controls. However, SC-144 reduced RV expression of GP130, STAT3, pSTAT3, and the pSTAT3/STAT3 ratio to near control levels (FIGS. 12A-12B). Immunofluorescence analysis of RV free wall sections showed increased cardiomyocyte GP130 membrane localization (FIG. 12C) and pSTAT3 positive nuclei (FIG. 12D) in MCT-V as compared to control. SC-144 decreased the immunoreactivity of both GP130 and pSTAT3 (FIGS. 12C-12D) when compared to MCT-V.

SC-144 Prevented Dysregulation of the MT-JPH2 Pathway and Adverse t-Tubule Remodeling in the RV

Next, we performed an in silico analysis of potential STAT3-regulated microtubule proteins by querying the human STAT3 ChIP-seq database. STAT3 was predicted to induce transcription of multiple tubulin isoforms and microtubule associated protein 4 (MAP4), a microtubule stabilizing protein (Table 4).

TABLE 4
Predicted STAT3 regulated microtubule proteins from
the ENCODE database.
STAT3 Regulated Microtubule Proteins

TUBA1A
TUBA1B
TUBA1C
TUBA4A
TUBA4B
TUBA8
TUBAL3
TUBB1
TUBB3
TUBB4B
TUBB6
TUBE1
TUBG1
TUBG2
MAP1LC3B
MAP1LC3B2
MAP1LC3C
MAP1S
MAP4
MAP7
MAP9
MAPRE2
MAPRE3
MAPT

In agreement with our in silico predictions, α-tubulin, β-tubulin, detyrosinated α-tubulin (tubulin found in stabilized microtubules) and MAP4 protein levels were elevated in MCT-V RV when compared to control (FIGS. 12E-12F). Microtubule remodeling resulted in downregulation of JPH2 in the MCT-V RV. SC-144 completely prevented upregulation of all tubulin isoforms and MAP4 and normalized JPH2 expression levels (FIGS. 12E-12F). We subsequently evaluated RV t-tubule architecture as a structural readout of altered JPH2 regulation. Control RV cardiomyocytes displayed a highly regular, striated t-tubule staining pattern, but MCT-V RV cardiomyocytes exhibited near complete loss of organized t-tubule structure (FIGS. 12G-12H). However, RV t-tubule morphology was restored with SC-144 (FIGS. 12G-12H).

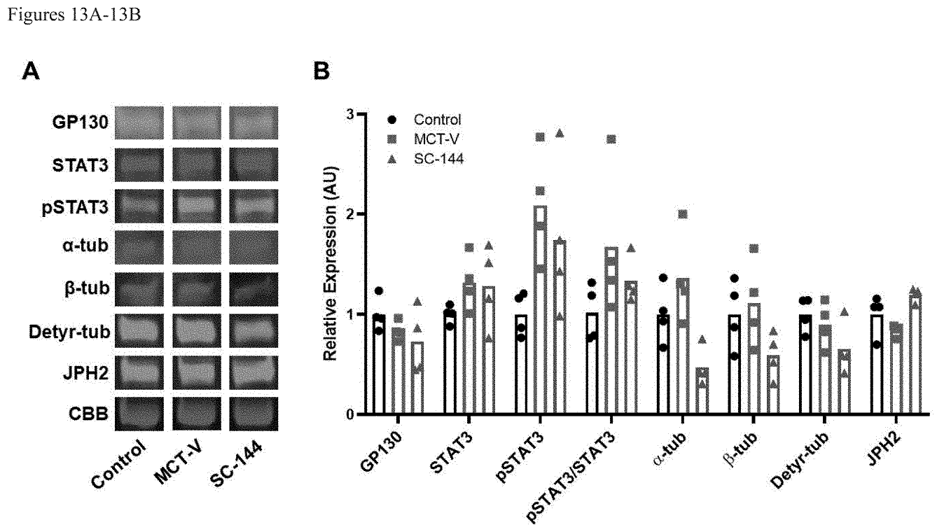

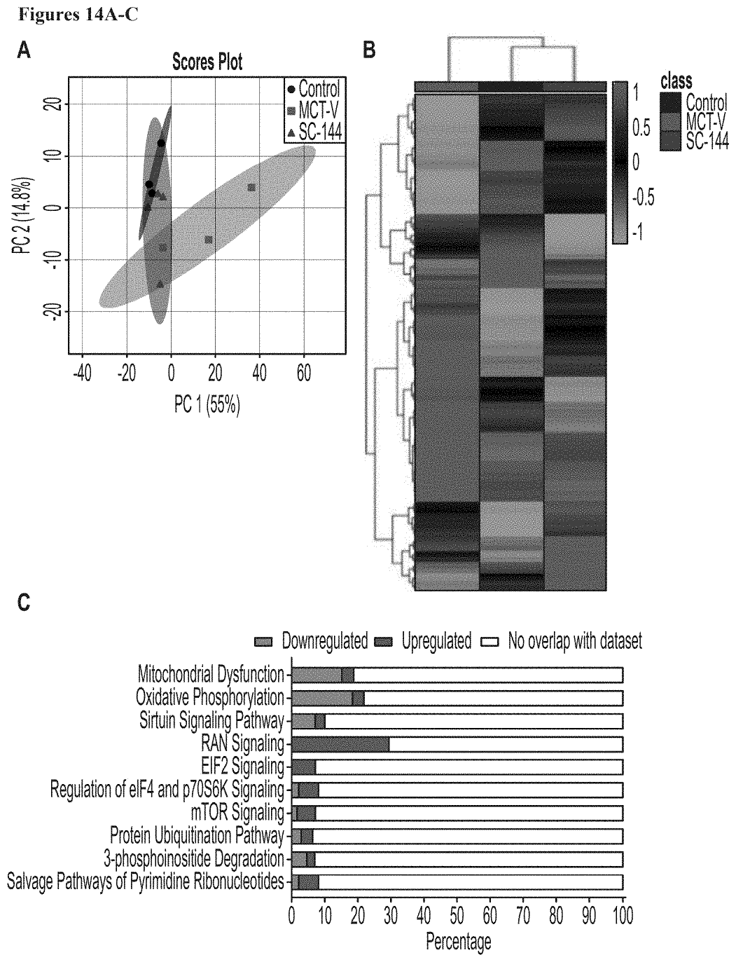

In contrast to the RV, the GP130 and MT-JPH2 pathways were minimally altered in MCT-V LV (FIGS. 13A-13B). Expression of GP130, STAT3, tubulins, and JPH2 were not different between MCT-V and control LV. pSTAT3 and the pSTAT3/STAT3 ratio were minimally elevated in MCT-V LV. SC-144 did not change the expression of GP130, STAT3, pSTAT3, pSTAT3/STAT3, or JPH2 in the LV. These results suggested GP130-mediated MT-JPH2 dysregulation is confined to the RV, and it is not a systemic effect of MCT.

Quantitative Proteomics Identified a Link Between Microtubule Remodeling and Mitochondrial Metabolic Dysregulation

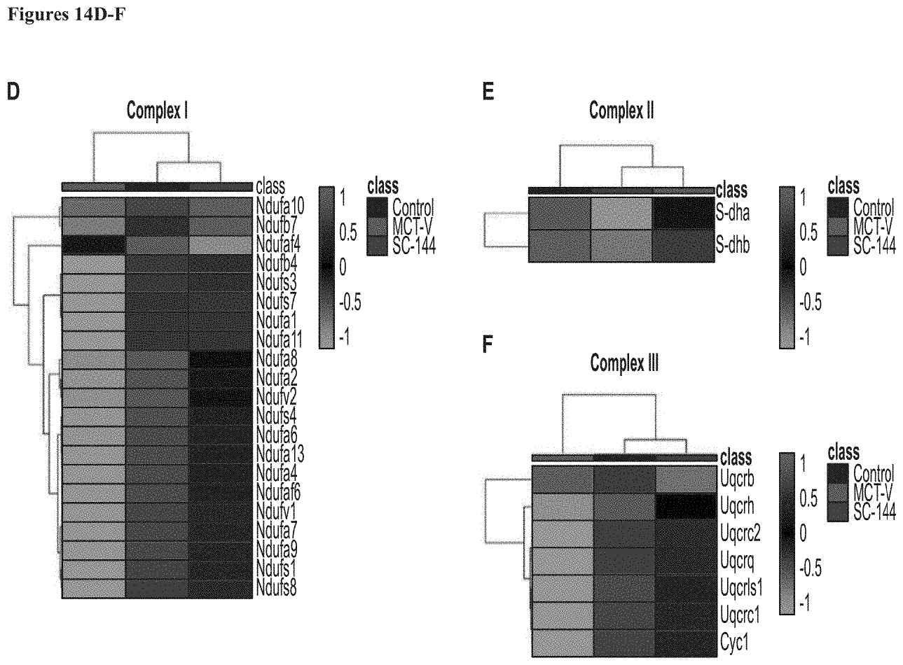

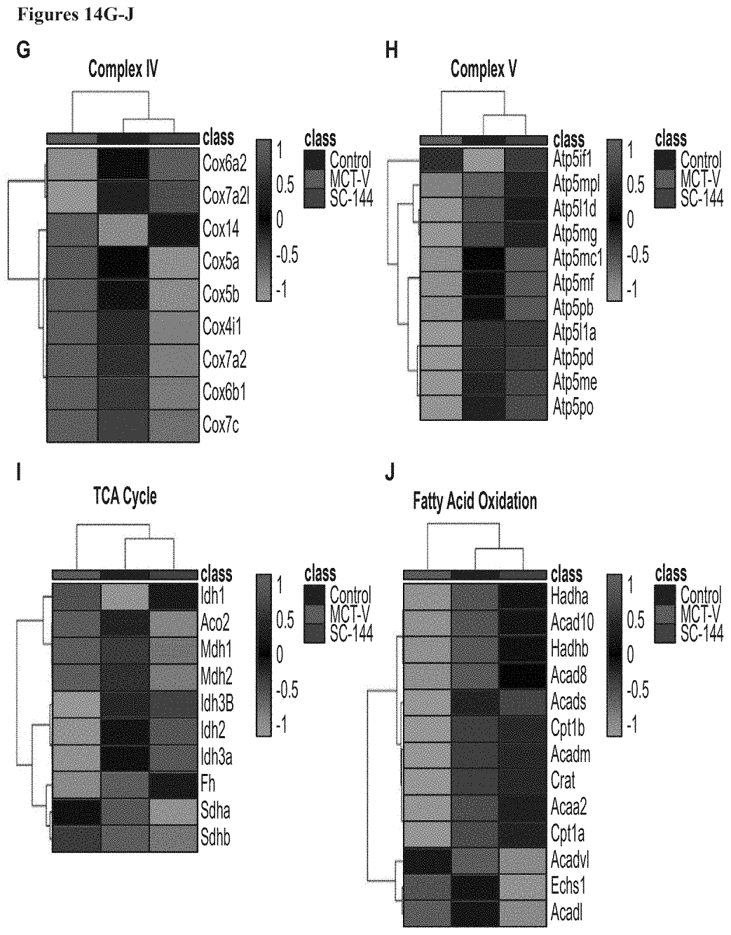

To delineate the effects of microtubule remodeling on other aspects of RV cardiomyocyte biology, we used quantitative mass spectrometry to define RV MAPs using a tissue-based microtubule cosedimentation assay. We identified 2854 MAPs in RV extracts and 1032 displayed significant variation in expression when comparing the three groups. In MCT rats, GP130 antagonism shifted the RV MAP signature towards control as depicted by principal component (FIG. 14A) and hierarchical cluster analyses (FIG. 14B). Ingenuity pathway analysis of the dysregulated MAPs revealed mitochondrial dysfunction and oxidative phosphorylation were the two most significantly enriched pathways (FIG. 14C). Hierarchical cluster analysis demonstrated altered regulation of proteins in complexes I-V of the electron transport chain, the tricarboxylic acid (TCA) cycle, and the fatty acid oxidation pathway in MCT-V RVs. SC-144 normalized expression levels of nearly all of these key mitochondrial metabolic proteins (FIGS. 14D-14J). Collectively, these data suggested microtubule remodeling in the MCT-V RV modulated mitochondrial homeostasis, and SC-144 corrected these pathological changes.

GP130 Antagonism Corrected RV Metabolism

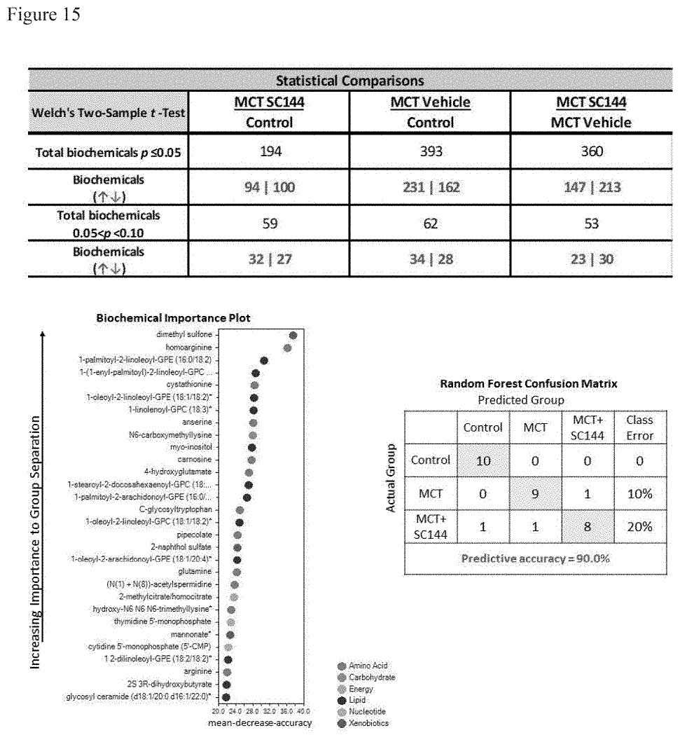

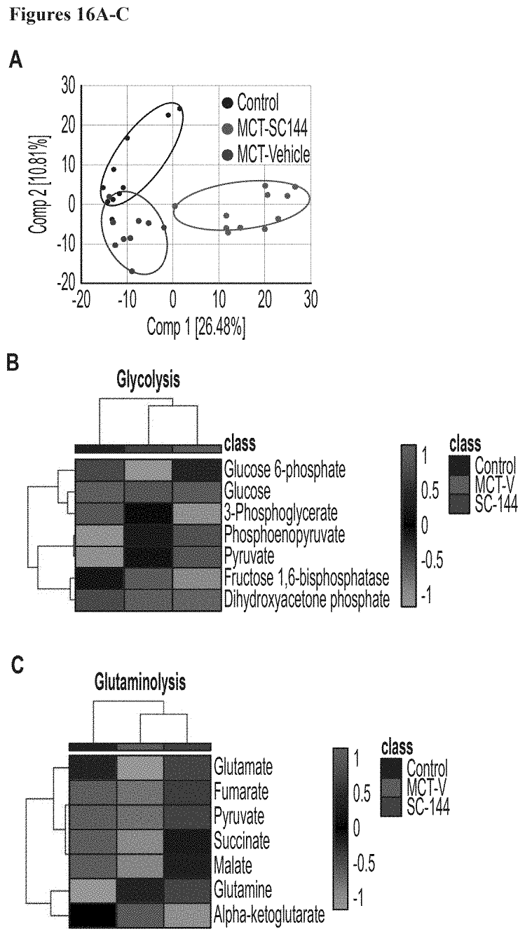

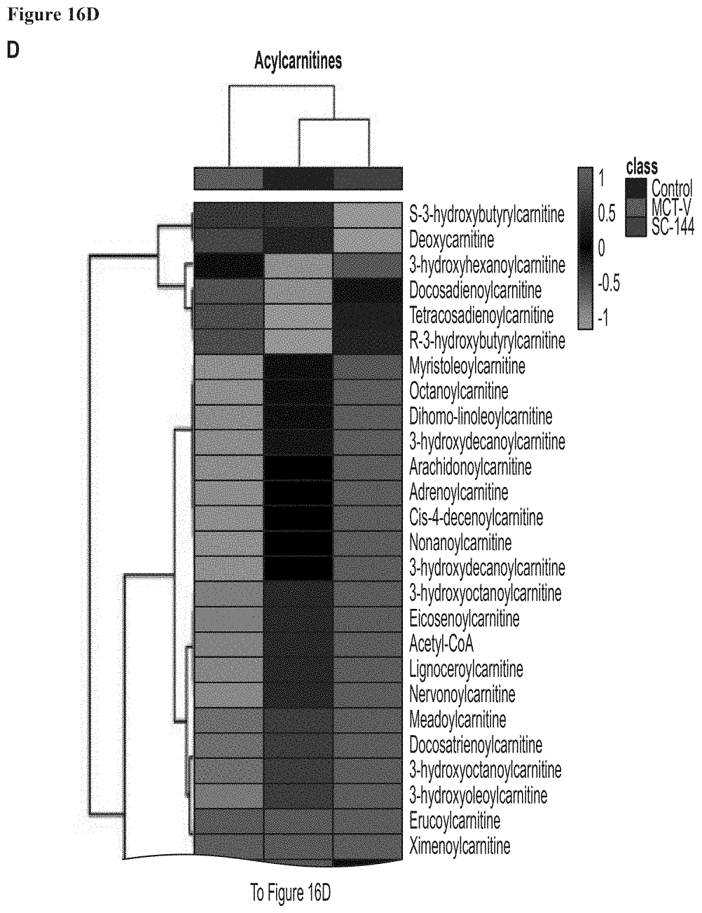

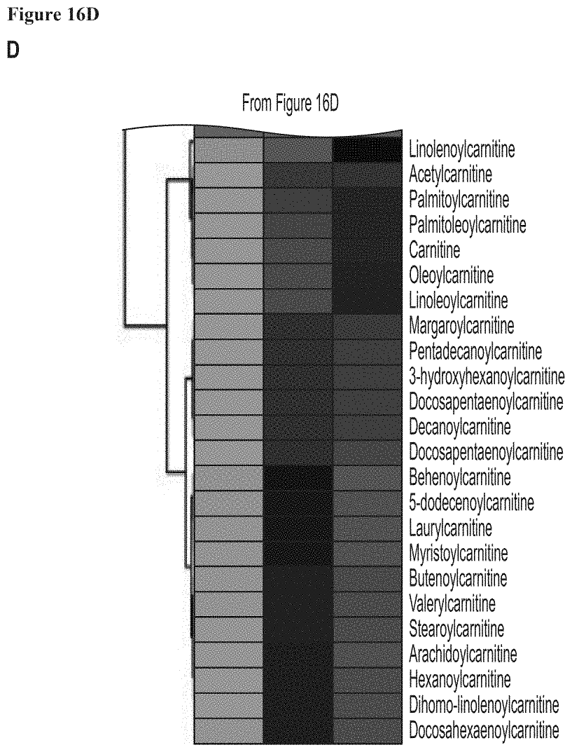

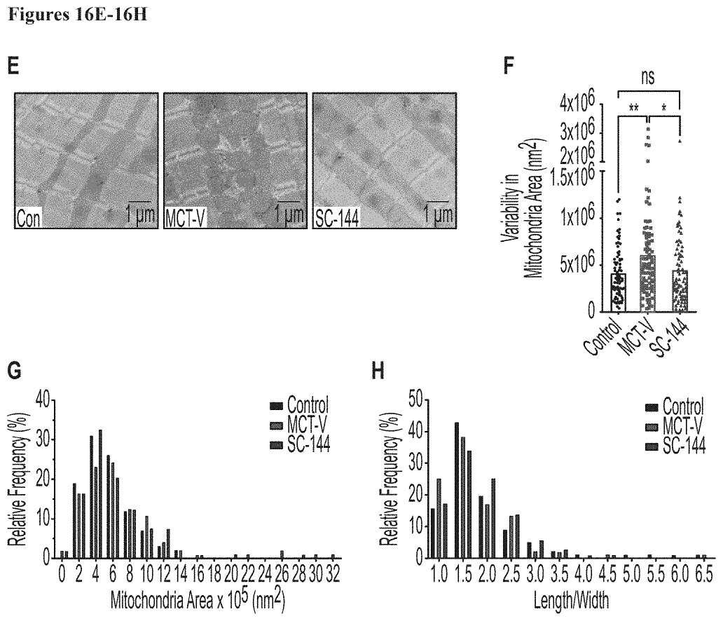

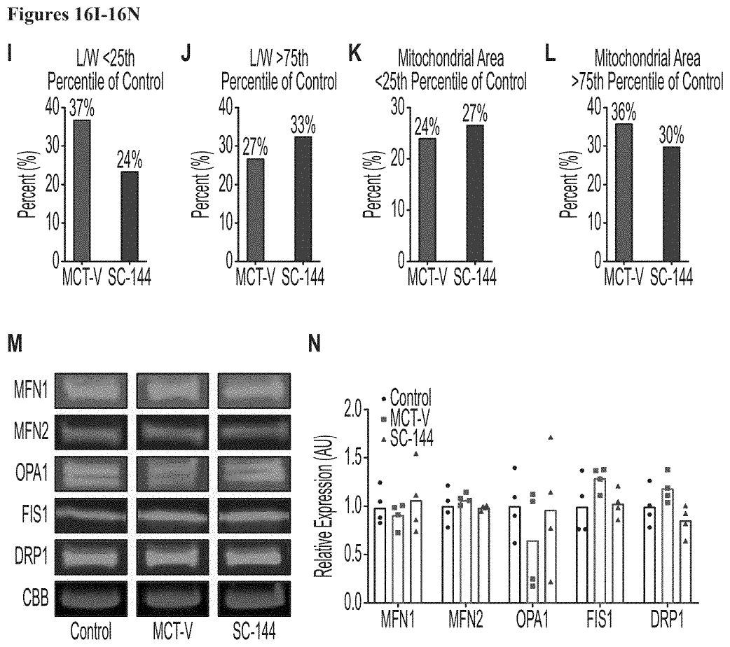

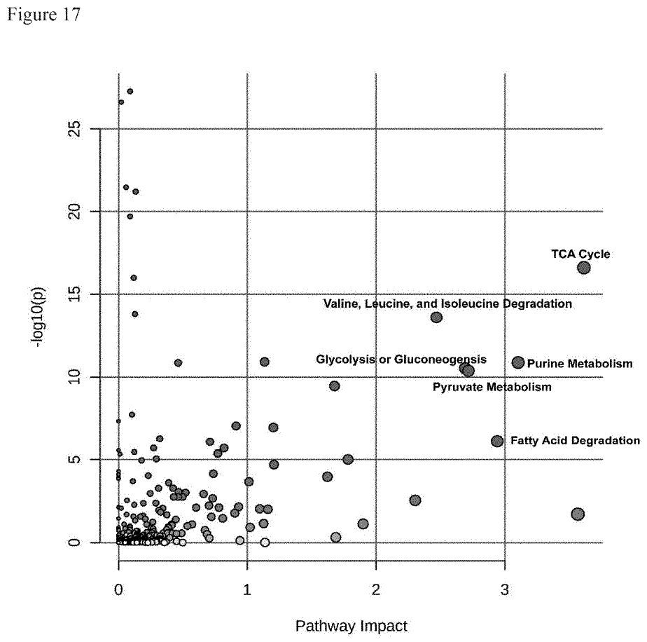

Global metabolomic profiling of 767 metabolites in RV free wall specimens defined the impact of mitochondrial protein dysregulation on RV metabolism (FIG. 15). Control and MCT-V RVs exhibited distinct metabolic signatures, but SC-144 shifted the global RV metabolic profile towards control (FIG. 16A). In particular, metabolite profiling suggested increased glycolytic metabolism and glutaminolysis induction in the MCT-V RV, which SC-144 blunted (FIGS. 16B-16C). Furthermore, there was evidence of impaired fatty acid oxidation in the MCT-V RV as nearly all acylcarnitines examined were reduced. However, SC-144 normalized acylcarnitine abundance, suggestive of augmented fatty acid metabolism (FIG. 16D). Consistent with our targeted analysis, computational integration of our proteomics and metabolomics analyses identified the TCA cycle, purine metabolism, pyruvate metabolism, glycolysis or gluconeogenesis, and fatty acid degradation as the most altered metabolic pathways in the MCT-V RV (FIG. 17). In summary, these data showed GP130 antagonism corrected deficits in multiple metabolic pathways in the RV.

SC-144 Restored Mitochondrial Morphology Via Normalization of Fission/Fusion Balance

Next, we used TEM to examine RV cardiomyocyte mitochondrial morphology to determine if disruption of the mitochondrial fission/fusion balance contributed to the observed metabolic defects. As compared to control mitochondria, MCT-V mitochondria had a spherical shape and had more size variability marked by an increased abundance of both large and small mitochondria (FIGS. 16E-16F). These results are consistent with more mitochondrial fission in MCT-V RVs. SC-144 corrected mitochondrial morphology (FIG. 16E), evident by a reduction in mitochondrial size variability (FIGS. 16F-16G) and restored mitochondria ellipticity (FIGS. 16H-16J). Although the percentage of small mitochondria were similar between MCT-V and SC-144 (FIG. 16K), GP130 antagonism decreased the amount of large/swollen mitochondria (FIG. 16L).

To supplement our TEM analysis, we evaluated the effects of SC-144 on RV mitochondrial fission/fusion protein regulation. When compared to controls, MCT-V RV protein expression profile favored mitochondrial fission as there was reduced abundance of the pro-fusion protein, optic atrophy protein 1 (OPA1) and increased expression of the pro-fission proteins, mitochondrial fission 1 (FIS1) and dynamin-related protein 1 (DRP1) (FIGS. 16M-16N). There was no change of expression of the fusion mediators, mitofusin (MFN)-1 and MFN-2 in MCT-V RV. However, GP130 antagonism prevented downregulation of OPA1 and upregulation of both FIS1 and DRP1 (FIGS. 16M-16N). Thus, the summation of our TEM findings and protein expression changes suggested SC-144 rebalanced RV mitochondrial fission/fusion.

Microtubule Stabilization Impaired Mitochondrial Metabolism In Vitro

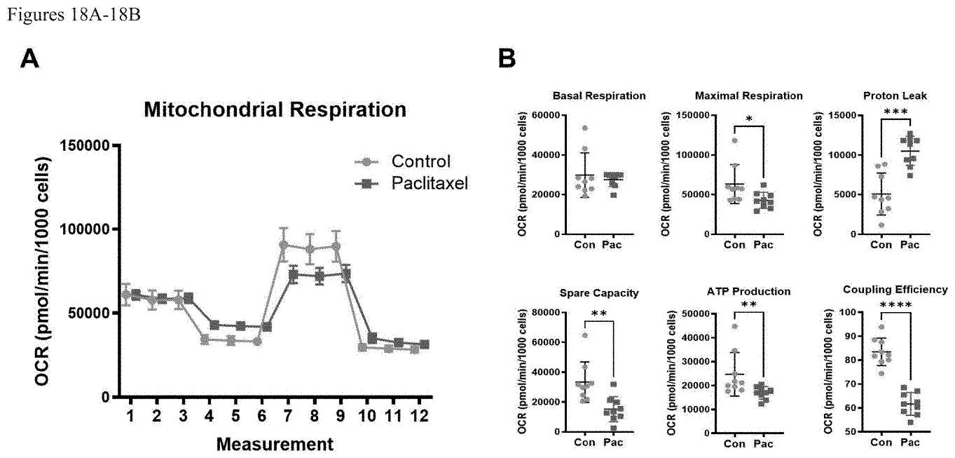

To examine the effects of microtubule remodeling on mitochondrial metabolic capacity, we quantified the effects of paclitaxel, a microtubule-stabilizing compound, on Seahorse micropolarimetry-defined mitochondrial function. Paclitaxel depressed H9c2 cardiomyocyte mitochondrial metabolic activity, evident in that maximal respiration, spare capacity, ATP production, and coupling efficiency were reduced and proton leak was enhanced (FIGS. 18A-18B). These results directly linked microtubule stability and mitochondrial metabolic dysfunction.

Inhibition of GP130 Signaling Did not Affect Pulmonary Vascular Disease

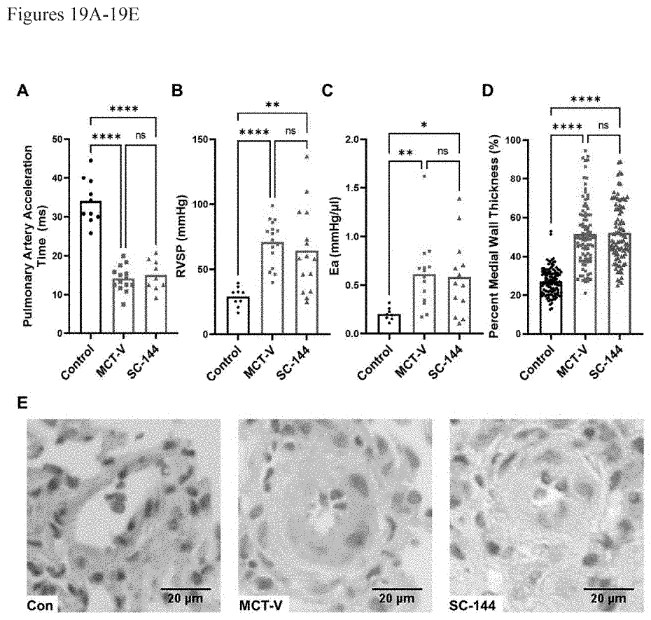

Because studies showed IL-6 promotes adverse pulmonary vascular remodeling, we quantified the effects of SC-144 on PAH severity. MCT-V and SC-144 rats displayed a nearly identical PAH phenotype, evident by similar pulmonary artery acceleration time (Control: 34.0±1.8, MCT-V: 14.1±0.9, SC-144: 15.0±1.3 ms, p=0.87 between MCT-V and SC-144), right ventricular systolic pressure (RVSP) (Control: 29.2±2.5, MCT-V: 71.0±4.3, SC-144: 64.5±8.4 mmHg, p=0.86 between MCT-V and SC-144), and effective arterial elastance (Ea) (Control: 0.35±0.04, MCT-V: 0.61±0.10, SC-144: 0.58±0.11 mmHg/μ1, p=0.995 between MCT-V and SC-144) (FIGS. 19A-19C). Furthermore, the percent medial thickness of small pulmonary arterioles was similarly increased in MCT-V and SC-144 rats as compared to controls (Control: 27.1±0.6, MCT-V: 51.6±1.7, SC-144: 52.0±1.6%, p=0.996 between MCT-V and SC-144) (FIGS. 19D-19E). Thus, SC-144 did not alter PAH severity in established PAH.

GP130 Antagonism Decreased RV Hypertrophy and Fibrosis

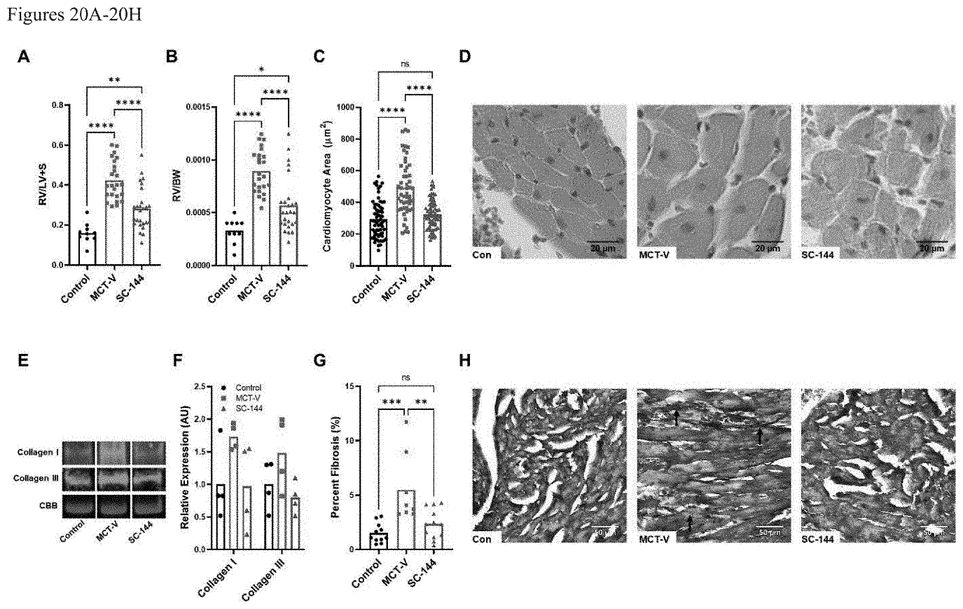

Next, we evaluated the effects of SC-144 on RV hypertrophy. Fulton index (RV/LV+S) (Control: 0.16±0.02, MCT-V: 0.42±0.02, SC-144: 0.28±0.02) and RV mass normalized to body mass (RV/BW) (Control: 0.33±0.04, MCT-V: 0.89±0.04, SC-144: 0.57±0.05 mg/g) were significantly reduced in the SC-144 rats compared to MCT-V (FIGS. 20A-20B). Furthermore, SC-144 significantly decreased cardiomyocyte cross-sectional area compared to MCT-V (Control: 295±14, MCT-V: 493±24, SC-144: 323±11 μm²) (FIGS. 20C-20D). However, SC-144 did not completely prevent RV hypertrophy as Fulton index and RV/BW were higher than control (FIGS. 20A-20H).

Then, we examined how SC-144 regulated RV fibrosis. MCT-V rats had higher RV collagen I and III protein levels than controls, but SC-144 prevented collagen I/III protein accumulation (FIG. 20E-20F). Likewise, GP130 antagonism reduced RV fibrosis on Masson's trichrome staining (Control: 1.5±0.3, MCT-V: 5.5±1.1, SC-144: 2.3±0.4%) (FIG. 20G-20H).

Inhibition of GP130 Signaling Enhanced RV Systolic Function and Survival

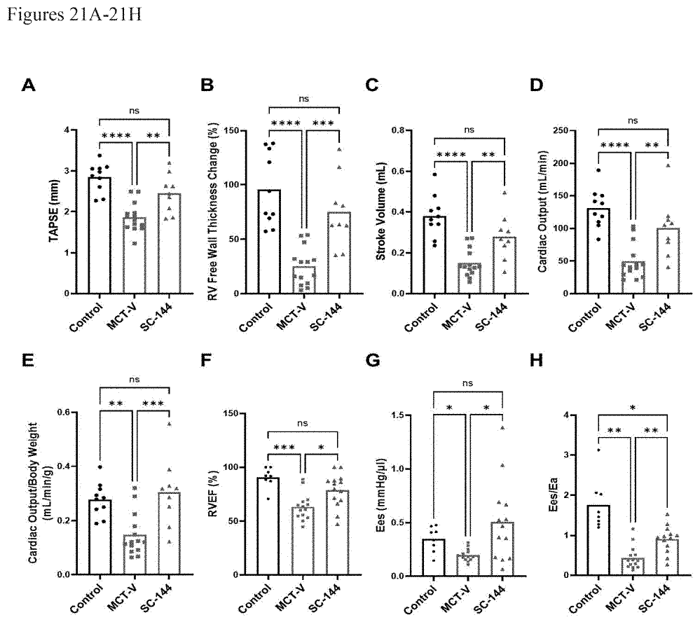

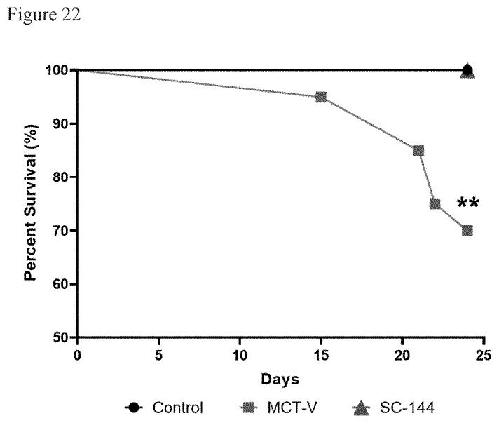

Both echocardiography and pressure-volume loop analysis demonstrated impaired RV systolic function in MCT-V rats as compared to controls (FIGS. 21A-21H). However, SC-144 augmented RV function as all the following parameters were higher in SC-144 than MCT-V rats: tricuspid annular plane systolic excursion (TAPSE) (Control: 2.8±0.1, MCT-V: 1.9±0.1, SC-144: 2.4±0.2 mm), percent change in RV free wall thickness (Control: 96±11, MCT-V: 25±5, SC-144: 75±11%), stroke volume (Control: 0.38±0.03, MCT-V: 0.15±0.02, SC-144: 0.28±0.04 mL), cardiac output (Control: 131±9, MCT-V: 49±7, SC-144: 101±15 mL/min), and cardiac output normalized to body weight (Control: 0.28±0.02, MCT-V: 0.15±0.02, SC-144: 0.31±0.04 mL/min/g) (FIGS. 21A-21E). Moreover, invasive hemodynamic studies showed GP130 antagonism improved RV ejection fraction (Control: 91±3, MCT-V: 63±3, SC-144: 79±4%), RV end-systolic elastance (Ees) (Control: 0.3±0.04, MCT-V: 0.2±0.02, SC-144: 0.5±0.1 mmHg/μ1), and RV-pulmonary artery coupling (Ees/Ea) (Control: 1.8±0.2, MCT-V: 0.4±0.1, SC-144: 0.9±0.1) (FIGS. 21F-21H). Finally, SC-144 prevented premature mortality (FIG. 22).

Elevated IL-6 Levels in PAH Patients were Associated with Worse RV Function

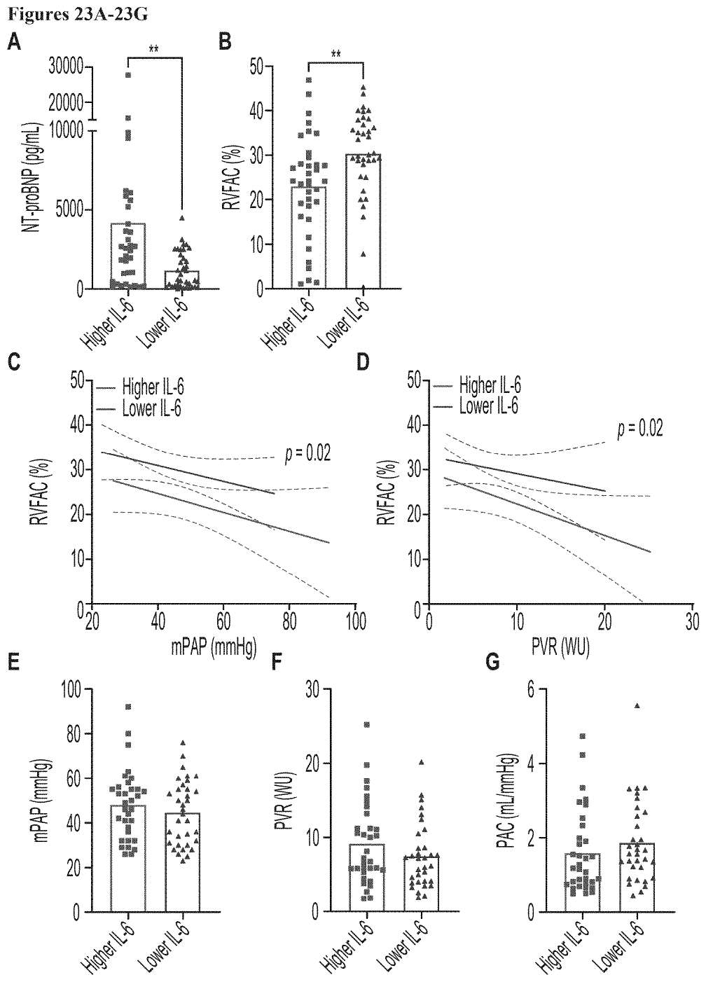

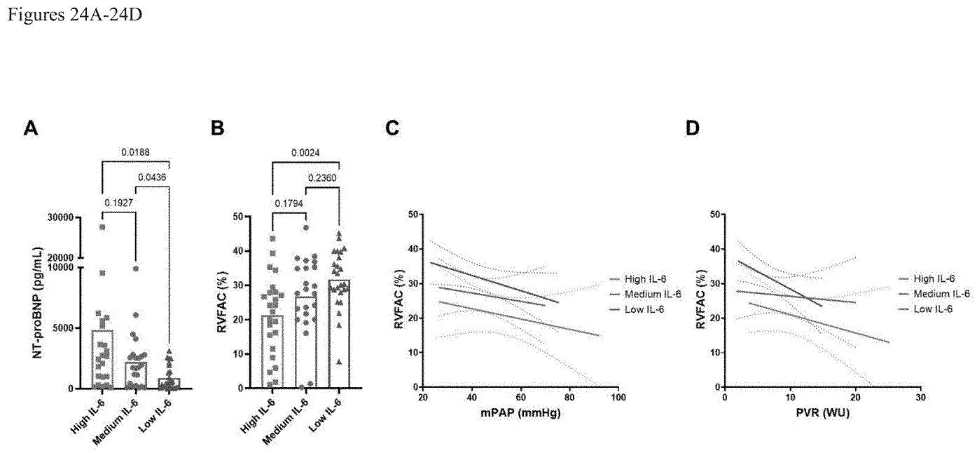

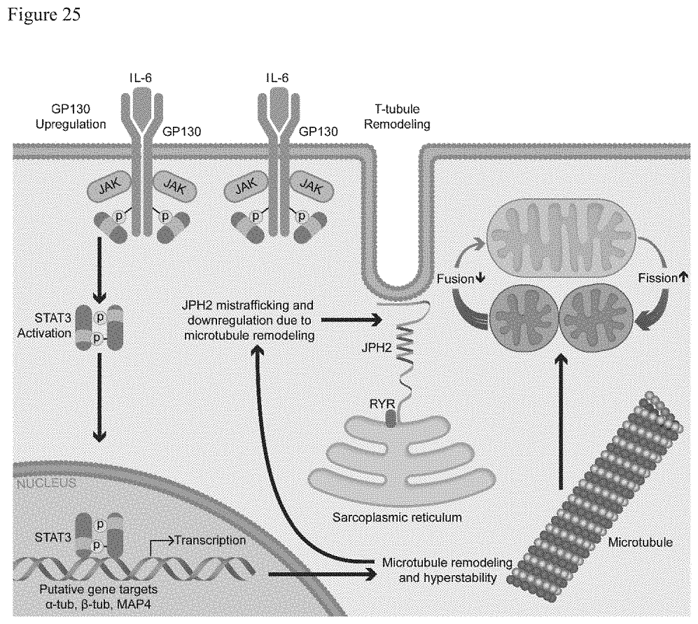

Finally, we addressed the translatability of our preclinical studies by examining how serum levels of the GP130 ligand, IL-6, were related to RV function in 73 PAH patients (Table 3). After dichotomizing our cohort by median IL-6 level, patients with elevated IL-6 levels had higher N-terminal pro B-type natriuretic peptide levels (NT pro-BNP) (median level 2576 vs. 599 pg/ml, p=0.001) and lower RV fractional area change (RVFAC) (23±2 vs. 30±2%, p=0.005) (FIGS. 23A-23B). When we plotted the relationship between RVFAC and mean pulmonary arterial pressure (mPAP), patients with higher IL-6 levels had lower RVFAC at all corresponding mPAPs (p=0.02) (FIG. 23C). Likewise, patients with high IL-6 levels had decreased RVFAC at all pulmonary vascular resistance (PVR) values (p=0.02) (FIG. 23D). These results suggested IL-6 had negative RV inotropic properties. Consistent with an RV-predominant effect of IL-6, there were no significant differences in mPAP (48±3 vs. 45±2 mmHg, p=0.35), PVR (9.2±1.0 vs. 7.5±0.8 Wood units, p=0.18), and pulmonary arterial compliance (1.6±0.2 vs. 1.9±0.2 mL/mmHg, p=0.32) between the two groups (FIGS. 23E-23G). Lastly, when we divided our cohort into tertiles, patients with the highest IL-6 levels had elevated NT-proBNP levels and reduced RVFAC compared to the middle and lowest IL-6 tertiles (FIGS. 24A-24B). When we defined the relationships between RVFAC and measures of RV afterload (mPAP and PVR), patients with the highest IL-6 levels had the lowest predicted RVFAC of the three tertiles (FIG. 24C-24D).

Discussion

In this study, we demonstrate GP130 signaling causes many of the observed pathological subcellular changes in the PAH RV (microtubule remodeling, JPH2 downregulation, t-tubule derangements, and mitochondrial dysfunction) that cause RVD (25). Small molecule antagonism of GP130 mitigates STAT3 activation, which restores expression of α- and β-tubulin, detyrosinated α-tubulin, and MAP4. The normalization of the microtubule cytoskeleton reverses pathological t-tubule remodeling, restores mitochondrial morphology and metabolic function, decreases RV hypertrophy, enhances RV function, and improves survival. This study also establishes the effects of the GP130 pathway on mitochondrial metabolism. Dysregulation of proteins in complexes I-V of the electron transport chain, the TCA cycle, and the fatty acid oxidation pathway in MCT-V RVs occurred, consistent with increased glycolytic metabolism and glutaminolysis and impaired fatty acid oxidation. The metabolic changes were all improved by GP130 antagonism. Importantly, all of these molecular and physiological changes occur independently of pulmonary vascular disease severity and were chamber specific, demonstrating a RV-specific effect of GP130 antagonism. Finally, in PAH patients, elevated serum levels of the GP130 agonist, IL-6 are associated with more severe RVD, suggesting targeting GP130 signaling may have translational utility.

The importance of the MT-JPH2 pathway and t-tubule remodeling in cardiac dysfunction are well-documented. Microtubule-mediated JPH2 disorganization causes t-tubule disruption and LV failure in rodent pressure-overload. Furthermore, overexpression of JPH2 prevents loss of t-tubules and improves LV function in pressure-overloaded mice. JPH2 is not as extensively studied in RVD, but JPH2 expression is reduced by 35% in MCT RV cardiomyocytes. Additionally, sildenafil, which restores RV JPH2 expression and t-tubule architecture, improves RV contractility in MCT rats. Moreover, we previously showed colchicine reduces microtubule density and increases JPH2 expression, which augments RV function. Collectively, these findings highlight the crucial role of JPH2 for proper LV and RV function. However, in PAH, the LV is relatively unaffected and thus the GP130-STAT3-JPH2 microtubular pathway is primarily dysregulated in the RV.

The direct link between GP130 signaling and microtubule remodeling may be pleiotropic as there are multiple mechanisms by which the GP130-STAT3 axis could modulate MT dynamics. As discussed above, STAT3 regulates expression of multiple isoforms of α- and β-tubulin and MAP4 (Table 4), which would promote microtubule stability and adverse remodeling. In addition, STAT3 can directly bind and stabilize microtubules in vitro. However, our proteomics analysis did not reveal differences in STAT3 levels in the MAP fraction between MCT-V and SC-144. Moreover, prior immunofluorescence studies do not show significant co-localization of STAT3 and microtubules in cardiomyocytes. Thus, the transcriptional modulation of tubulin isoforms and MAP4 via STAT3 is a more likely mechanism underlying the observed, GP130-dependent cardiomyocyte microtubule stabilization. However, PI3K and MAPK signaling may also alter the microtubule cytoskeleton and these pathways merit future investigation.

Our finding that GP130 modulates RV fibrosis is congruent with multiple previous publications. First, genetic deletion of IL-6 reduces interstitial fibrosis in diabetic cardiomyopathy. Moreover, inhibition of STAT3 activity with parthenolide decreases LV fibrosis via modulation of fibroblast activation. Additionally, β_{IV}-spectrin knockout in fibroblasts activates STAT3 signaling and causes cardiac fibrosis and dysfunction. Importantly, these pathological changes are reversed by pharmacological inhibition of STAT3. Thus, there are multiple lines of evidence linking the GP130-STAT3 axis to pathological cardiac fibrosis, which likely explains our finding that SC-144 decreases RV fibrosis (FIGS. 20A-20H).

Our proteomics/metabolomics analyses and Seahorse experiments demonstrate that microtubules can modify mitochondrial metabolism. This hypothesis is supported by other publications. In particular, microtubules are proposed to be critical hubs for mitochondrial movement and activity as metabolites and mitochondria can be enriched in discrete subcellular locations via microtubule-mediated trafficking. Additionally, microtubules facilitate inter-mitochondrial interactions in cardiomyocytes, which are important for maintenance of proper cardiac function. Finally, in yeast, the association of mitochondria with microtubules prevents mitochondrial fission by inhibition of binding of Dnm1, the yeast analogue of DRP1. This alters mitochondrial fission/fusion balance and ultimately mitochondrial function. In summary, there are strong data showing the microtubule cytoskeleton can modulate mitochondrial metabolic function.

The correction of multiple metabolic pathways with SC-144 further validates the pathogenic effects of impaired metabolism on RV function. Consistent with a previous study, we show glutaminolysis is activated in the MCT-V RV (FIGS. 16A-16N). The attenuation of glutaminolysis with SC-144, and the fact that pharmacological inhibition of glutaminolysis enhances RV function in MCT rats demonstrates this metabolic pathway has a maladaptive effect on RV function. Importantly, glutaminolysis is also induced in human RVD, so this has direct human relevance. In addition, we show alteration of fatty acid metabolism is associated with RVD. MCT-V rats have reduced RV acylcarnitine and acetyl Co-A levels (surrogate measures of fatty acid metabolism), but those metabolites are normalized with SC-144 (FIGS. 16A-16N). The finding of impaired fatty acid oxidation in the PAH RV is consistent with our prior work in fawn hooded rats, which develop spontaneous pulmonary hypertension and RV failure. Interestingly, human PAH RV samples also have decreased acylcarnitines levels. Thus, our data and the human metabolomics study support an important role for fatty acid metabolism in proper RV function. In conclusion, the ability of SC-144 to combat glutaminolysis induction and enhance fatty acid metabolism, two pathways also altered in human RVD, highlights the importance of metabolism for proper RV function.

The fact that GP130 antagonism did not alter the pulmonary vasculature might seem to contradict the findings of Tamura et al. (Tamura Y, Phan C, Tu L, Le Hiress M, Thuillet R, Jutant E M, Fadel E, Savale L, Huertas A, Humbert M, Guignabert C. Ectopic upregulation of membrane-bound il6r drives vascular remodeling in pulmonary arterial hypertension. J Clin Invest. 2018; 128:1956-1970). This group used an IL-6 receptor/soluble IL-6 receptor antagonist, 20S,21-epoxy-resibufogenin-3-formate, and they showed it lowered mPAP and attenuated adverse pulmonary vascular remodeling in MCT rats. However, their molecule more exclusively targets IL-6, and the differences in timing of treatment may explain why our results are dissimilar. Tamura et al. started therapy one week after MCT, whereas we initiated therapy two weeks after MCT, a timepoint when PAH is more established. Consistent with our findings, TRANSFORM-UK (NCT02676947) showed IL-6-specific inhibition with tocilizumab does not alter PVR, suggesting IL-6 inhibition does not usually reverse established pulmonary hypertension. Moreover, our study along with Soon et al. (Soon E, Holmes A M, Treacy C M, Doughty N J, Southgate L, Machado R D, Trembath R C, Jennings S, Barker L, Nicklin P, Walker C, Budd D C, Pepke-Zaba J, Morrell N W. Elevated levels of inflammatory cytokines predict survival in idiopathic and familial pulmonary arterial hypertension. Circulation. 2010; 122:920-927) and Simpson et al. (Simpson C E, Chen J Y, Damico R L, Hassoun P M, Martin L J, Yang J, Nies M, Griffiths M, Vaidya R D, Brandal S, Pauciulo M W, Lutz K A, Coleman A W, Austin E D, Ivy D D, Nichols W C, Everett A D. Cellular sources of interleukin-6 and associations with clinical phenotypes and outcomes in pulmonary arterial hypertension. Eur Respir J. 2020; 55) do not show any relationship between serum IL-6 levels and pulmonary vascular hemodynamics in PAH. Perhaps the association between elevated IL-6 levels and mortality in PAH may be due to more severe RV dysfunction, and not heightened pulmonary vascular disease.

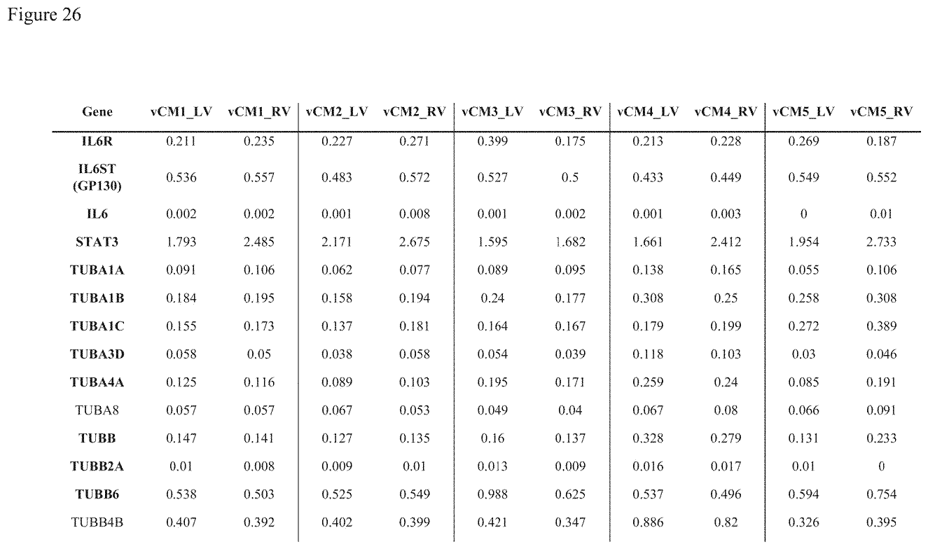

Finally, the pathogenic effects of GP130 signaling may be more relevant to RVD than LV dysfunction as analysis of the publicly available human heart single nucleus RNA sequencing database shows expression of GP130, STAT3, and many tubulin isoforms are higher in RV cardiomyocytes than LV cardiomyocytes (FIG. 26). We speculate that this pathway may be more active in the RV, and thus blocking the GP130-microtubule axis may have accentuated beneficial effects in RVD as compared to LV failure.

Conclusions

Inhibition of GP130 signaling enhances RV function independent of changes in the pulmonary vasculature. Modulation of pathological microtubule remodeling with GP130 antagonism leading to enhanced t-tubule structure and normalization of mitochondrial metabolism likely underlies the improved RV function.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

When used in this specification and the claims as an adverb rather than a preposition, “about” means “approximately” and comprises the stated value and every non-negative value within 10% of that value; in other words, “about 100%” includes 90% and 110% and every value in between. Unless stated otherwise, every range or interval includes both endpoints and every value in between.

As used herein the term “consisting essentially of” is defined to mean that specified materials may optionally be included in the composition that do not materially affect the basic and novel characteristics of the claims. Examples of such materials include preservatives and dispersants that do not have an impact on the function of the therapeutic composition.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of treating right ventricular dysfunction in pulmonary hypertension by administering a first composition comprising a GP130 antagonist in a patient in need thereof.

2. The method of claim 1, wherein the treating improves right ventricular function by at least about 10%.

3. The method of claim 1, wherein the treating improves right ventricular function by at least about 20%.

4. The method of claim 1, wherein the treating improves right ventricular function by at least about 30%.

5. The method of claim 1, wherein the treating improves right ventricular function by at least about 40%.

6. The method of claim 1, wherein the IL-6 antagonist targets an IL-6 co-receptor.

7. The method of claim 6, wherein the IL-6 co-receptor is GP130.

8. The method of claim 1, wherein the IL-6 antagonist is SC-144.

9. The method of claim 1, wherein the administration is by oral administration.

10. The method of claim 1, wherein the administration is by intravenous infusion.

11. The method of claim 1, wherein the pulmonary hypertension is pulmonary arterial hypertension (PAH), pulmonary hypertension owing to left heart disease, pulmonary hypertension due to chronic lung disease, chronic thromboembolic pulmonary hypertension (CTEPH), or pulmonary hypertension with unclear multifactorial mechanisms.

12. A method of treating an elevated blood level of IL-6 by administering a first composition comprising a GP130 antagonist in a patient in need thereof.

13. The method of claim 12, wherein the IL-6 antagonist targets an IL-6 co-receptor.

14. The method of claim 13, wherein the IL-6 co-receptor is GP130.

15. The method of claim 12, wherein the IL-6 antagonist is SC-144.

16. The method of claim 12, wherein the administration is by oral administration.

17. The method of claim 12, wherein the administration is by intravenous infusion.

18. The method of claim 1, wherein the treatment further comprises administering a second composition comprising a phosphodiesterase inhibitor, endothelin receptor antagonist, soluble guanylate cyclase activator, IP3 receptor agonist, or prostacyclin.

19. The method of claim 18, wherein the first and second compositions are administered simultaneously.

20. The method of claim 18, wherein the first and second compositions are administered sequentially.

21. The method of claim 18, wherein the first and second compositions are administered separately.
